(19) United States
(12) Patent Application Publication
(10) Pub. No.: US 2025/0058118 A1
(43) Pub. Date: Feb. 20, 2025

(54) MANAGING OBSTRUCTIVE SLEEP APNEA THROUGH AIRWAY NEUROSTIMULATION

(71) Applicant: Restora Medical, Inc., Irvine, CA (US)

(72) Inventor: Quan Ni, Irvine, CA (US)

(21) Appl. No.: 18/742,879

(22) Filed: Jun. 13, 2024

Related U.S. Application Data
(60) Provisional application No. 63/519,463, filed on Aug. 14, 2023.

(51) Int. Cl. A61N 1/36

(52) U.S. Cl. CPC A61N 1/3611; A61N 1/36135; A61N 1/36146

(57) ABSTRACT

At least some embodiments of the present disclosure are directed to systems and methods for managing sleep disordered breathing (e.g., obstructive sleep apnea) for a patient. An implantable electrode delivers a stimulation signal proximate to a nerve of the patient to stimulate the nerve and activate at least one muscle associated with an airway of the person. A controller generates a set of predicted stimulation settings for the stimulation signal using a trained machine learning model and provides the set of predicted stimulation settings to the stimulation signal generator.

### CROSS-REFERENCE TO RELATED PATENT APPLICATIONS

This application claims priority to U.S. Provisional Application No. 63/519,463, filed on Aug. 14, 2023, the disclosure of which is hereby incorporated herein by reference in its entirety.

### BACKGROUND

Obstructive sleep apnea (OSA) is a common sleep-related breathing disorder. OSA may occur when the muscles in the back of the throat relax during sleep, causing a partial or complete blockage of the airway. Typical treatment options may include positive airway pressure (PAP) therapy using devices like continuous positive airway pressure (CPAP) to help keep the airway open during sleep, and oral appliances using custom-made mouthpieces to reposition the jaw and tongue to prevent airway collapse.

### SUMMARY

Certain embodiments of the present disclosure relate to medical systems, apparatus, and methods for managing sleep disordered breathing (e.g., obstructive sleep apnea) in a patient. More specifically, some embodiments of the present disclosure relate to medical systems, apparatus, and methods for managing obstructive sleep apnea in a patient through airway neurostimulation.

According to some embodiments, a system for managing obstructive sleep apnea for a person includes an implantable electrode configured to deliver a stimulation signal proximate to a nerve of the person to stimulate the nerve and activate at least one muscle of the person, and a stimulation signal generator configured to deliver the stimulation signal to the implantable electrode. The stimulation signal includes a series of stimulation cycles each including a stimulation period and a non-stimulation period. A controller is functionally connected to the stimulation signal generator to control operation of the stimulation signal generator, and the controller is configured to apply a trained machine learning model to a set of initial stimulation settings for the stimulation signal to generate a set of predicted stimulation settings for the stimulation signal.

According to certain embodiments, a method for managing obstructive sleep apnea for a person includes providing an implantable electrode configured to deliver a stimulation signal proximate to a nerve to stimulate the nerve and activate at least one muscle of the person; applying a trained machine learning model to a set of initial stimulation settings for the stimulation signal to generate a set of predicted stimulation settings for the stimulation signal; and delivering the stimulation signal to the implantable electrode. The stimulation signal has a series of stimulation cycles each including a stimulation period and a non-stimulation period.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1A illustrates a schematic diagram representing patient anatomy and an example medical system for airway neurostimulation, in accordance with embodiments of the present disclosure.

FIG. 1B illustrates a schematic diagram representing the patient anatomy of FIG. 1A.

FIG. 2 is a block diagram of an example medical system for managing obstructive sleep apnea of a patient through airway neurostimulation, in accordance with embodiments of the present disclosure.

FIG. 3 is an example flow diagram of a medical method for managing obstructive sleep apnea of a patient through airway neurostimulation, in accordance with embodiments of the present disclosure.

FIG. 4A is a schematic diagram illustrating example timing cycles of dual neurostimulation in a first mode, in accordance with embodiments of the present disclosure.

FIG. 4B is a schematic diagram illustrating example timing cycles of dual neurostimulation in a second mode, in accordance with embodiments of the present disclosure.

FIG. 5A is a schematic diagram illustrating example neuromodulation entrainment with natural breathing rhythm, in accordance with embodiments of the present disclosure.

FIG. 5B is an example flow diagram of a medical method for managing obstructive sleep apnea of a patient through airway neurostimulation, in accordance with embodiments of the present disclosure.

FIGS. 6A-C are example flow diagrams illustrating titration methods, in accordance with some embodiments of the present disclosure.

FIG. 7A illustrates a schematic diagram representing patient anatomy and example target muscle location(s) for hypoglossal nerve stimulation, in accordance with embodiments of the present disclosure.

FIG. 7B illustrates a schematic diagram representing patient anatomy and example target muscle location(s) for ansa cervicalis stimulation, in accordance with embodiments of the present disclosure.

FIG. 7C illustrates a schematic diagram representing patient anatomy and example target nerve/muscle location(s) for an airway neurostimulation including hypoglossal nerve stimulation and ansa cervicalis stimulation, in accordance with embodiments of the present disclosure.

FIG. 7D illustrates a schematic diagram representing patient anatomy and example target nerve/muscle location(s) for ansa cervicalis stimulation in an upper airway dual neurostimulation, in accordance with embodiments of the present disclosure.

FIGS. 8A and 8B are example flow diagrams of methods for determining stimulation settings for managing obstructive sleep apnea of a patient through upper airway neurostimulation, in accordance with embodiments of the present disclosure.

FIG. 9 is a simplified block diagrams of an example computing device, with which aspects of the present disclosure may be practiced.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

The following detailed description is exemplary in nature and is not intended to limit the scope, applicability, or configuration of the disclosure in any way. Rather, the following description provides some practical illustrations for implementing exemplary embodiments of the present disclosure. Examples of constructions, materials, and/or dimensions are provided for selected elements. Those skilled in the art will recognize that many of the noted examples have a variety of suitable alternatives.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g., 1 to 5 includes 1, 1.5, 2, 2.75, 3, 3.80, 4, and 5) and any number within that range.

Although illustrative methods may be represented by one or more drawings (e.g., flow diagrams, communication flows, etc.), the drawings should not be interpreted as implying any requirement of, or particular order among or between, various steps disclosed herein. However, some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information. In some embodiments, the term “receive” or “receiving” means obtaining from a data repository (e.g., database), from another system or service, from another software, or from another software component in a same software. In certain embodiments, the term “access” or “accessing” means retrieving data or information, and/or generating data or information.

Typical treatment options for obstructive sleep apnea (OSA) in a patient such as positive airway pressure (PAP) therapy may not be suitable for many patients. Hypoglossal nerve stimulation (HNS) is considered as an effective form of therapy for patients with OSA for whom PAP therapy is not suitable. In a typical method for stimulating airway patency-related tissue using hypoglossal nerve stimulation (HNS), an implanted neurostimulator is used to generate stimulation signals to deliver to an implanted simulation lead based on respiratory information of the patient sensed by a sensing lead. Hypoglossal nerve stimulation (HNS) therapy can work by protruding and stiffening the tongue muscle thereby reducing obstruction from the tongue. However, HNS therapy may not reduce obstruction from the lateral wall. It was found that about 35% implanted patients do not respond to the hypoglossal nerve stimulation (HNS). For example, it was found that patients with lateral wall collapse had a reduced response to HNS. As such, ways to improve treatment for OSA are needed. At least some embodiments of the present disclosure are directed to medical systems, devices, and methods for managing obstructive sleep apnea in a patient through upper airway dual neurostimulation.

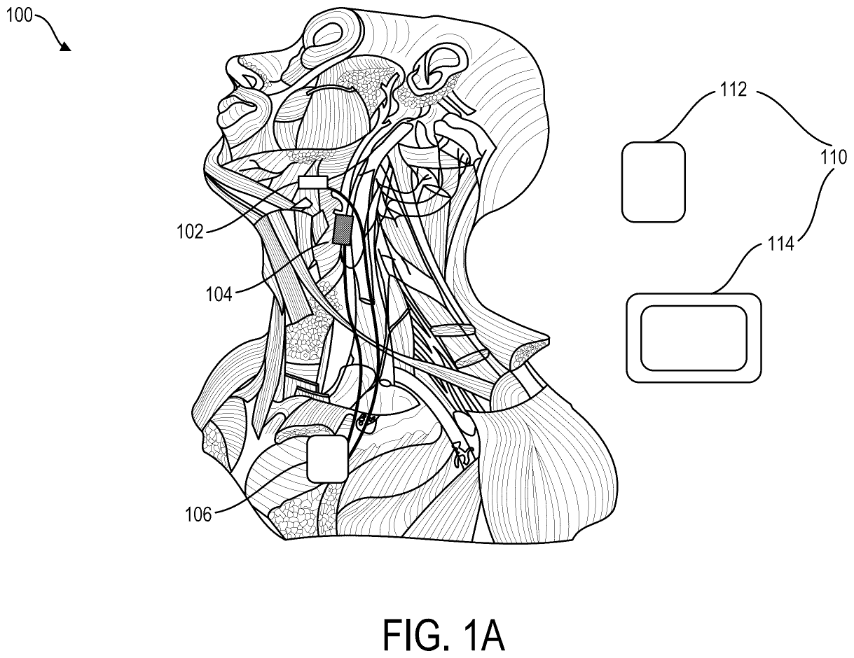

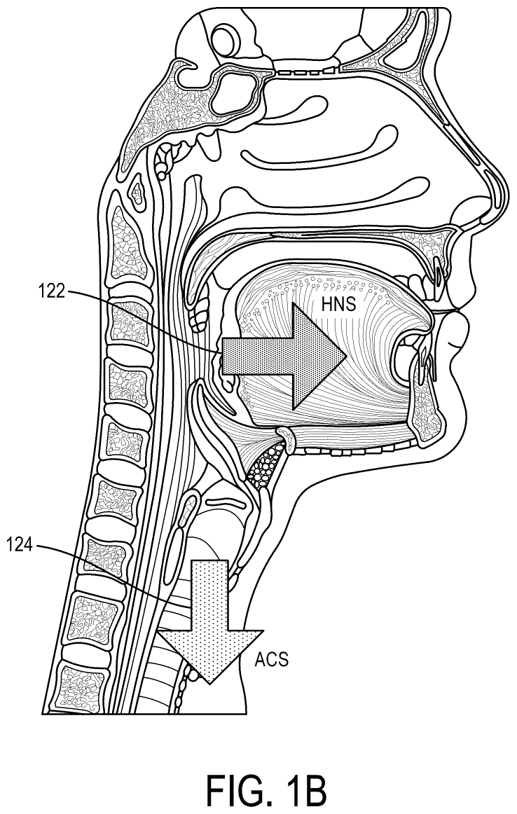

FIG. 1A illustrates a schematic diagram representing patient anatomy and a system 100 for upper airway dual neurostimulation for a person, in accordance with embodiments of the present disclosure. The system 100 includes a first implantable electrode 102, also referred to as a stimulation lead, configured to deliver a first stimulation signal proximate to a first nerve of the person to stimulate the first nerve and activate at least one muscle for an upper airway dilation of the person. In some embodiments, the system 100 further includes a second implantable electrode 104 configured to deliver a second stimulation signal proximate to a second nerve to stimulate the second nerve and activate at least one muscle for a caudal tracheal traction for an upper airway of the person. In certain embodiments, the system 100 includes only one of the first implantable electrode 102 and the second implantable electrode 104.

In some embodiments, the system 100 further includes a stimulation signal generator 106 configured to deliver the first stimulation signal to the first implantable electrode 102 and/or deliver the second stimulation signal to the second implantable electrode 104. The stimulation signal generator 106 can be positioned (e.g., implanted) in any suitable locations and be connected to the first and second implantable electrodes 102 and 104 wirelessly or via wires. In some embodiments, the stimulation signal generator 106 is implanted in or on a patient's body, for example, in the chest, adjacent to the implantable electrodes 102 and 104. In some embodiments, the implantable electrodes 102 and 104 can be operably coupled to the same, single stimulation signal generator 106. In some embodiments, the stimulation signal generator 106 can include a first stimulation signal generator coupled to the first implantable electrode 102 and a second stimulation signal generator coupled to the second implantable electrode 104. The first and second stimulation signal generators can be disposed within the same physical housing or separate housings.

According to certain embodiments, the stimulation signal generator 106 can be controlled to generate one or more stimulation signals and deliver the generated stimulation signals to one or more electrodes. In some embodiments, the stimulation signal generator 106 can be controlled to convey various patterns of electrical currents and voltages to generate the stimulation signals.

In some embodiments, the stimulation signal generator 106 can generate a first stimulation signal having a first set of stimulation parameters, and/or a second stimulation signal having a second set of stimulation parameters. In certain embodiments, the first stimulation signal has a series of first stimulation cycles each including a first stimulation period and a first non-stimulation period. In some embodiments, the second stimulation signal has a series of second stimulation cycles each including a second stimulation period and a second non-stimulation period.

In some embodiments, the stimulation signal generator 106 can be a pulse generator to generate a series of pulses in the stimulation period of each stimulation cycle. The stimulation signal generator 106 can control the one or more stimulation parameters of the pulse signal, including one or more of an amplitude, a frequency, a pulse width, a rate of amplitude change, a duty cycle, and the like.

In some embodiments, the stimulation signal generator 106 can coordinate the delivery of the first stimulation signal with the delivery of the second stimulation signal. In certain embodiments, the stimulation signal generator 106 includes an internal timer to provide timing for the series of first stimulation cycles of the first stimulation signal and the series of second stimulation cycles of the second stimulation signal. In some embodiments, the internal timer can synchronize the first stimulation periods and the first non-stimulation periods of the first stimulation signal with the second stimulation periods and the second non-stimulation periods of the second stimulation signal, respectively.

In some embodiments, the system 100 further includes a controller 110 functionally connected to the stimulation signal generator 106 to control operation of the stimulation signal generator. In some embodiments, the controller 110 is configured to control or adjust one or more stimulation parameters for the stimulation signal generator 106 including, for example, a duration of stimulation cycle, a duration of a stimulation period, a duration of a non-stimulation period, a coordination between a first stimulation signal and a second stimulation signal, a pulse amplitude, a pulse frequency, a pulse width, a duty cycle of the generated stimulation signal, and the like. In some embodiments, the controller 110 allows a user to adjust a first amplitude of the first stimulation signal and/or a second amplitude of the second stimulation signal to obtain an optimized or predicted combination of the first amplitude and the second amplitude.

In the embodiment depicted in FIG. 1A, the controller 110 includes a first controller 112 and a second controller 114. In some embodiments, the first controller 112 can be a patient remote controller for a patient to control operation of the stimulation signal generator 106. For example, the patient can use the controller to turn on or turn off the stimulation signal generator 106, to adjust the respective amplitudes of one or more stimulation signals, to switch the operation of the stimulation signal generator 106 between a first mode and a second mode, and the like. In some embodiments, the controller 110 can automatically turn on or off the stimulation signal generator 106 based on the time of day. In some embodiments, the second controller 114 can be a clinician programming device for a physician or clinician to adjust one or more stimulation parameters for the stimulation signal generator 106.

In some embodiments, the first implantable electrode 102 is configured to deliver the first stimulation signal proximate to a hypoglossal nerve to stimulate the hypoglossal nerve and activate at least one tongue muscles. In some embodiments, the second implantable electrode 104 is configured to deliver a second stimulation signal proximate to an ansa cervicalis nerve to stimulate the ansa cervicalis nerve and activate at least one infrahyoid muscle.

FIG. 1B illustrates a schematic diagram representing the patient anatomy of FIG. 1A showing a treatment effect through upper airway dual neurostimulation. In some embodiments, the first stimulation signal is delivered to activate one or more protrusor muscles of the tongue (e.g., genioglossus) to displace tongue base anteriorly as indicated by arrow 122 of FIG. 1B for hypoglossal nerve stimulation (HNS), which can pull soft palate anteriorly and stiffen the pharyngeal lateral wall antero-posteriorly. In some embodiments, the second stimulation signal is delivered to activate one or more infrahyoid muscles to descend a hyoid-thyroid complex, which results in a trachea caudal traction that stiffens the pharyngeal lateral wall and posterior wall inferiorly as indicated by arrow 124 of FIG. 1B for ansa cervicalis nerve stimulation (ACS).

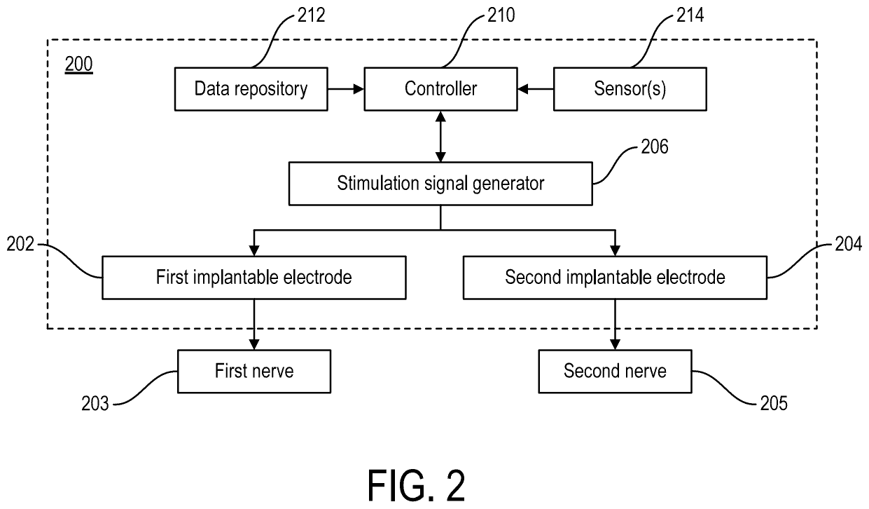

FIG. 2 is a block diagram of a medical system 200 for managing obstructive sleep apnea in a patient through upper airway neurostimulation, in accordance with embodiments of the present disclosure.

In some embodiments, the medical system 200 includes a first implantable electrode 202 configured to deliver a first stimulation signal proximate to a first nerve 203 of the person to stimulate the first nerve 203, a second implantable electrode 204 configured to deliver a second stimulation signal proximate to a second nerve 205. In some embodiments, the medical system 200 can include one of the first implantable electrode 202 and the second implantable electrode 204.

In some embodiments, the first implantable electrode 202 is configured to deliver the first stimulation signal proximate to a hypoglossal nerve to stimulate the hypoglossal nerve and activate at least one tongue muscle. In some embodiments, the second implantable electrode 204 is configured to deliver a second stimulation signal proximate to an ansa cervicalis nerve to stimulate the ansa cervicalis nerve and activate at least one infrahyoid muscle. In some embodiments, the medical system 200 can include a third implantable electrode configured to deliver a third stimulation signal proximate to a third nerve to stimulate the third nerve. The third nerve can be, for example, a phrenic nerve.

In some embodiments, the medical system 200 further includes a stimulation signal generator 206 configured to deliver the first stimulation signal to the first implantable electrode 202 and deliver the second stimulation signal to the second implantable electrode 204. The first stimulation signal has a series of first stimulation cycles including a first stimulation period and a first non-stimulation period. The second stimulation signal has a series of second stimulation cycles including a second stimulation period and a second non-stimulation period. The delivery of the first stimulation signal is coordinated with the delivery of the second stimulation signal.

In some embodiments, the stimulation signal generator 206 can include one or more pulse generators each configured to generate a stimulation signal including a series of stimulation cycles including a stimulation period and a non-stimulation period. A stimulation period of a stimulation cycle can include a series of stimulation pulses having one or more pulse parameters. Example pulse parameters include a pulse frequency, an amplitude, a pulse width, a duty cycle, and the like. A pulse frequency can be, for example, from about 5 Hz to about 40 Hz (e.g., at or about 30 Hz). A pulse width can be, for example, from about 10 microseconds to about 1000 microseconds (e.g., at or about 100 microseconds). A duty cycle can refer to a percentage of a duration of stimulation at a pulse amplitude to a duration of a stimulation cycle (e.g., the sum of a duration of stimulation and a duration of no stimulation). A duration of a stimulation cycle can be, for example, in the range from 2 seconds to 10 minutes. A duty cycle can be, for example, in a range from about 5 percent to 95 percent. A pulse amplitude may refer to the difference between a higher voltage level and a lower voltage level. A pulse amplitude can be, for example, in the range from 0.1 to 15 volts or 0.1 to 15 mA.

In some embodiments, the medical system 200 further includes a controller 210 functionally connected to the stimulation signal generator 206 to control operation of the stimulation signal generator. In some embodiments, the controller 210 is a remote controller configured to control one or more stimulation parameters for the stimulation signal generator 106 including, for example, one or more of an amplitude, a frequency, a pulse width, a rate of amplitude change, a duty cycle, and the like, of the generated stimulation signal.

In some embodiments, the controller 210 can include a patient remote controller for a patient to control operation of the stimulation signal generator 206 including, for example, to turn on or off the stimulation signal generator 206, to adjust the respective amplitudes of one or more stimulation signals, to switch the operation of the stimulation signal generator 106 from a first mode to a second mode, and the like.

In some embodiments, the controller 210 can include a clinician programming device for a physician or clinician to pre-program the stimulation signal generator 206 with desired stimulation parameters. The stimulation parameters can be controllable to allow one or more stimulation signals be remotely modulated to desired settings without removal of the corresponding electrodes from their target positions.

In some embodiments, the controller 210 can include one or more computing devices each of which can include a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the medical system 200 further includes one or more sensors 214. In certain embodiments, the sensors 214 can be external sensors configured to detect one or more physiological information of the patient, including, for example, the apnea-hypopnea index (AHI), the oxygen desaturation index (ODI), the respiratory disturbance index (RDI), a posture change, a sleep stage, a body motion for restlessness, and the like. It is to be understood that the external sensors can be any suitable type of sensors including, for example, an acoustic sensor for snoring detection. In some embodiments, apnea-hypopnea index (AHI) refers to a measure of the number of times a person or patient has upper airway obstruction during sleep. For example, an AHI of five to fifteen can be a mild sleep apnea. The patient's AHI can be monitored by using any suitable sensors/devices. Similarly, ODI and RDI measures oxygen saturation and respiratory flow changes resulted from upper airway obstruction associated with sleep apnea.

According to certain embodiments, the sensors 214 can generate a sensor signal based on the physiological parameters or changes and send the sensor signal to the controller 210. The controller 210 can process the sensor signal and control/adjust one or more stimulation parameters of the stimulation signal generator 206 based, at least in part, on the one or more physiological parameters. In some examples, the sensors 214 can include a position sensor to sense body position or posture during sleep. In some examples, the sensors 214 can determine a sleep stage, for example, whether the patient is in a deep sleep or a shallow sleep. In some examples, multiple sensors can be combined to measure the patient's AHI during sleep.

In some embodiments, the sensors 214 can send the related sensing data to the controller 210 to determine whether a patient is entering a stable sleep. When the controller 210 determines that the patient is entering a stable sleep, the controller 210 can retrieve a stored therapy setting from the data repository and send the therapy setting to the stimulation signal generator 206 to adjust the corresponding one or more first stimulation parameters of the first stimulation signal and/or one or more first stimulation parameters of the second stimulation signal.

In some embodiments, the sensors 214 can send the related sensing data to the controller 210 to determine whether a patient is turning to a supine posture. When the controller 210 determines that the patient is turning to a supine posture, the controller 210 can retrieve a stored therapy setting from the data repository and send the therapy setting to the stimulation signal generator 206 to adjust the corresponding one or more first stimulation parameters of the first stimulation signal and/or one or more first stimulation parameters of the second stimulation signal.

In some embodiments, the sensors 214 can send the related sensing data to the controller 210 to determine whether a patient is having an increased AHI. When the controller 210 determines that the patient is having an increased AHI, the controller 210 can retrieve a stored therapy setting from the data repository and send the therapy setting to the stimulation signal generator 206 to adjust the corresponding one or more first stimulation parameters of the first stimulation signal and/or one or more first stimulation parameters of the second stimulation signal.

In some embodiments, the medical system 200 further includes a data repository 212 to store data for the medical system 200. In some embodiments, the data repository 212 can be implemented using any one of the memory or storage configurations described below. A data repository can include random access memories, flat files, XML files, and/or one or more database management systems (DBMS) executing on one or more database servers or a data center. A database management system can be a relational (RDBMS), hierarchical (HDBMS), multidimensional (MDBMS), object oriented (ODBMS or OODBMS) or object relational (ORDBMS) database management system, and the like. The data repository can be, for example, a single relational database. In some cases, the data repository may include a plurality of databases that can exchange and aggregate data by a data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the medical system 200 can communicate via or be coupled to via a communication network or interface, for example, a wired or wireless network or interface. The communication network or interface can be any suitable communication network or combination of communication networks. For example, communication network can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard), a wired network, and the like. In some examples, communication network can be a local area network (LAN), a wide area network (WAN), a public network (e.g., the Internet), a private or semi-private network (e.g., a corporate or university intranet), any other suitable type of network, or any suitable combination of networks. Communication links (arrows) between components of the medical system 200 can each be any suitable communication link or combination of communication links, such as wired links, fiber optics links, Wi-Fi links, Bluetooth links, cellular links, and the like.

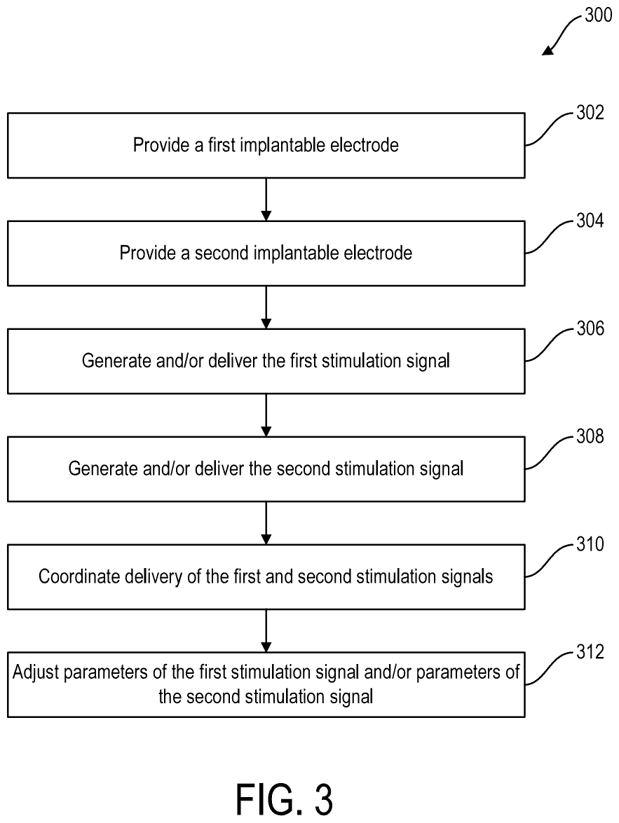

FIG. 3 is a flow diagram of a medical method 300 for managing obstructive sleep apnea (OSA) of a patient through upper airway neurostimulation, in accordance with embodiments of the present disclosure. The method is described in relation to the stimulation signal generators and electrodes discussed previously here. It is to be understood that any stimulation signal generators and electrodes can be used in the method. Aspects of embodiments of the method may be performed, for example, by a medical system or a controller (e.g., the medical system 200 in FIG. 2, the controller 210 in FIG. 2). One or more steps or blocks of method are optional and/or can be modified by one or more steps of other embodiments described herein. Additionally, one or more steps of other embodiments described herein may be added to the method.

According to certain embodiments, at process 302, a first implantable electrode is provided proximate to a first nerve of a patient. In some embodiments, the first implantable electrode can be implanted proximate to the hypoglossal nerve of the patient. In some embodiments, the first implantable electrode can be implanted proximate to one or more medial branches of the hypoglossal nerve (m-XII) to stimulate the one or more medial branches and activate one or more protrusion muscles of the at least one tongue muscle including genioglossus. In some embodiments, the first electrode can be transcutaneously implanted by inserting into a ranine vein of the patient to be placed and fixed proximate to the first nerve, which can be, for example, one or more medial branches of the hypoglossal nerve. In some embodiments, the first electrode can be percutaneously implanted by inserting through the skin and tissue of the patient to be placed and fixed proximate to the first nerve, which can be, for example, one or more medial branches of the hypoglossal nerve. In some embodiments, the nerve can be located proximate to a location that selectively recruits protrusion muscles and reduces recruitment of retraction muscles. In some examples, the first electrode can include one or more percutaneous electrodes, one or more cuff electrodes, one or more wire electrodes, and the like.

According to certain embodiments, at process 304, a second implantable electrode is provided proximate to a second nerve of a patient. In some embodiments, the second implantable electrode can be implanted to stimulate the ansa cervicalis nerve and activate one or more of the infrahyoid muscles including, for example, omohyoid, sternothyroid and sternohyoid muscles, combined muscles of omohyoid, sternothyroid and sternohyoid, or sternothyroid and sternohyoid. In some embodiments, the second implantable electrode can be implanted to deliver the second stimulation signal to stimulate certain nerve branch(es) of the ansa cervicalis and activate sternothyroid and sternohyoid muscles simultaneously. In some embodiments, the second electrode can be transcutaneously implanted by inserting into an internal jugular vein of the patient to placed and fixed to the second nerve to stimulate the ansa cervicalis nerve and activate one or more of the infrahyoid muscles including, for example, omohyoid, sternothyroid and sternohyoid muscles, combined muscles of omohyoid, sternothyroid and sternohyoid, or sternothyroid and sternohyoid. In some embodiments, the second electrode can be percutaneously implanted by inserting through the skin and tissue of the patient to be placed and fixed proximate to the second nerve to stimulate the ansa cervicalis nerve and activate one or more of the infrahyoid muscles including, for example, omohyoid, sternothyroid and sternohyoid muscles, combined muscles of omohyoid, sternothyroid and sternohyoid, or sternothyroid and sternohyoid. In some examples, the second electrode can include one or more percutaneous leads, one or more paddle leads, one or more cuff leads, and the like.

According to certain embodiments, at process 306, the stimulation signal generator 206 generates and/or delivers a first stimulation signal. In some embodiments, the first stimulation signal can include a series of first stimulation cycles including a first stimulation period and a first non-stimulation period. A first stimulation period of a stimulation cycle can include a series of first stimulation pulses having one or more first pulse parameters. Example first pulse parameters include a first pulse frequency, a first pulse amplitude, a first pulse width, a first duty cycle, and the like.

According to certain embodiments, at process 308, the stimulation signal generator 206 generates and/or delivers a second stimulation signal. In some embodiments, the second stimulation signal can include a series of second stimulation cycles each including a second stimulation period and a second non-stimulation period. A second stimulation period of a stimulation cycle can include a series of first stimulation pulses having one or more second pulse parameters. Example second pulse parameters include a second pulse frequency, a second pulse amplitude, a second pulse width, a second duty cycle, and the like.

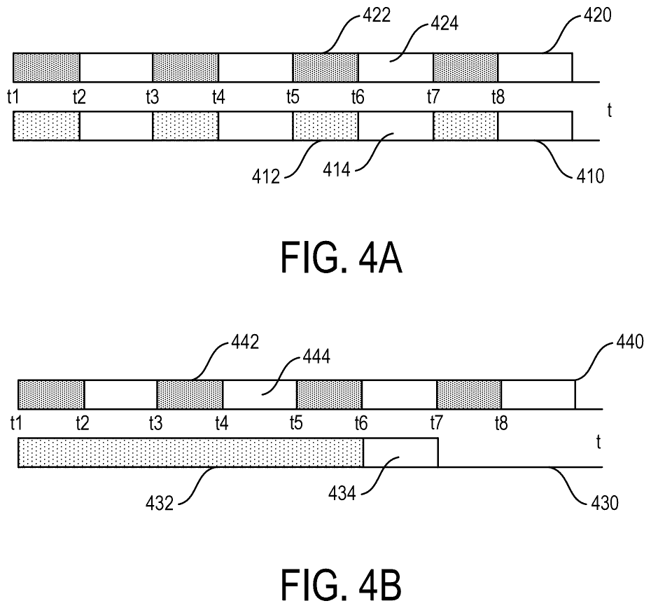

According to certain embodiments, at process 310, the controller 210 coordinates the delivery of the first stimulation signal through the first implantable electrode proximate to a first nerve and the delivery of the second stimulation signal through the second implantable electrode proximate to a second nerve. In some embodiments, the first nerve is stimulated to activate at least one muscle for an upper airway dilation of the patient. In some embodiments, the second nerve is stimulated to activate at least one muscle for a caudal tracheal traction for an upper airway of the patient. Examples of coordinating the delivery of the first and second stimulation signals are illustrated in FIGS. 4A and 4B, which will be described further below.

According to certain embodiments, at process 312, the controller 210 adjusts one or more first parameters of the first stimulation signal, and/or one or more second parameters of the second stimulation signal. In some embodiments, the controller 210 can control or adjust one or more stimulation parameters for the stimulation signal generator 206 to generate first and second stimulation signals. The stimulation parameters include, for example, a first/second duration of a first/second stimulation cycle of the first/second stimulation signal, a first/second duration of a first/second stimulation period of the first/second stimulation signal, a first/second duration of a first/second non-stimulation period of the first/second stimulation signal, a coordination between the first stimulation signal and the second stimulation signal, a first/second pulse amplitude of the first/second stimulation signal, a first/second pulse frequency of the first/second stimulation signal, a first/second pulse width of the first/second stimulation signal, a first/second duty cycle of the of the first/second stimulation signal, and the like. In some embodiments, the controller 210 allows a user to adjust a first amplitude of the first stimulation signal and/or a second amplitude of the second stimulation signal to obtain an optimized or predicted combination of the first amplitude and the second amplitude, while maintaining other stimulation parameters for the first and second stimulation signals. In some embodiments, the controller 210 allows a user to adjust the coordination of delivering the first and second stimulation signals by, for example, adjusting the onset/offset of the respective stimulation cycles.

FIG. 4A is a schematic diagram illustrating example timing cycles of dual neurostimulation in a first mode, in accordance with embodiments of the present disclosure. According to some embodiments, the stimulation signal generator operates in a first mode to synchronize the first stimulation periods and the first non-stimulation periods of the first stimulation signal with the second stimulation periods and the second non-stimulation periods of the second stimulation signal, respectively. As illustrated in the embodiment of FIG. 4A, a first stimulation signal 410 includes a series of stimulation signal cycle each including a first stimulation period 412 and a first non-stimulation period 414. A second stimulation signal 420 includes a series of stimulation signal cycle each including a second stimulation period 422 and a second non-stimulation period 424. In some embodiments, the duration T1_on of the first stimulation period 412 and the duration T2_on of the second stimulation period 422 can be substantially the same. That is, T1_on=T2_on. In some embodiments, the first stimulation signal 410 and the second stimulation signal 420 are substantially synchronized with each other. In some examples, the onset of the first stimulation period 412 and the onset of the second stimulation period 422 can be aligned with each other to occur concurrently (e.g., at t1, t3, t5, t7 . . . in FIG. 4A). In some examples, the onset of the first stimulation period 412 and the onset of the second stimulation period 422 can be aligned with each to have an offset T_{offset}. For example, when the onset of the first stimulation period 412 is at t1, the onset of the second stimulation period 422 can be at t1′=t1±T_{offset}.

FIG. 4B is a schematic diagram illustrating example timing cycles of dual neurostimulation in a second mode, in accordance with embodiments of the present disclosure. According to some embodiments, when the stimulation signal generator operates in a second mode, first stimulation signal(s) can be dominant compared to second stimulation signal(s). As illustrated in the embodiment of FIG. 4B, a first stimulation signal 430 includes a series of stimulation signal cycle each including a first stimulation period 432 and a first non-stimulation period 434. A second stimulation signal 440 includes a series of stimulation signal cycle each including a second stimulation period 442 and a second non-stimulation period 444. In some embodiments, the duration T1_on of the first stimulation period 432 can be greater than the duration T2_on of the second stimulation period 442. The ratio of T1_on over T2_on can be in a range, for example, from about 1.0 to about 200.0, from about 1.5 to about 200.0, or from about 2.0 to about 200.0. In one example, T2_on is 3 seconds, and T1_on is 10 minutes or 600 seconds.

In some embodiments, the dominant first stimulation signal(s) can be delivered proximate to a hypoglossal nerve to stimulate the hypoglossal nerve and activate at least one tongue muscle. In certain embodiments, the dominant first stimulation signal can be delivered proximate to one or more medial branches of the hypoglossal nerve (m-XII) to stimulate the one or more medial branches and activate one or more protrusion muscles of the at least one tongue muscle including genioglossus.

In some embodiments, the second stimulation signal(s) can be delivered proximate to an ansa cervicalis nerve to stimulate the ansa cervicalis nerve and activate one or more infrahyoid muscles. In some embodiments, the second stimulation signal(s) can stimulate two or more certain nerve branch(es) of ansa cervicalis simultaneously, for example, to activate sternothyroid and sternohyoid muscles simultaneously. In certain embodiments, the stimulation to activate the tongue protrusion muscles can be longer than the stimulation to activate the infrahyoid muscles since the former may have a less load.

In the embodiment depicted in FIG. 4B, the duration T1_on of the first stimulation period 432 substantially equates the sum of three times of duration T2_on of the second stimulation period 442 and two times of duration T2_off of the second non-stimulation period 444. That is, T1_on=3×T2_on+2×T2_off. In some embodiments, the duration T2_off of the second non-stimulation period 444 is substantially the same as the duration T1_off of the first non-stimulation period 434. The onset of one first stimulation period 432 and the onset T2_on of the second stimulation period 422 can be aligned with each other to occur concurrently (e.g., at t1). The offset of the one first stimulation period 432 of the first stimulation periods is synchronized (e.g., at t6) with an offset of another second stimulation period 442 of the second stimulation periods.

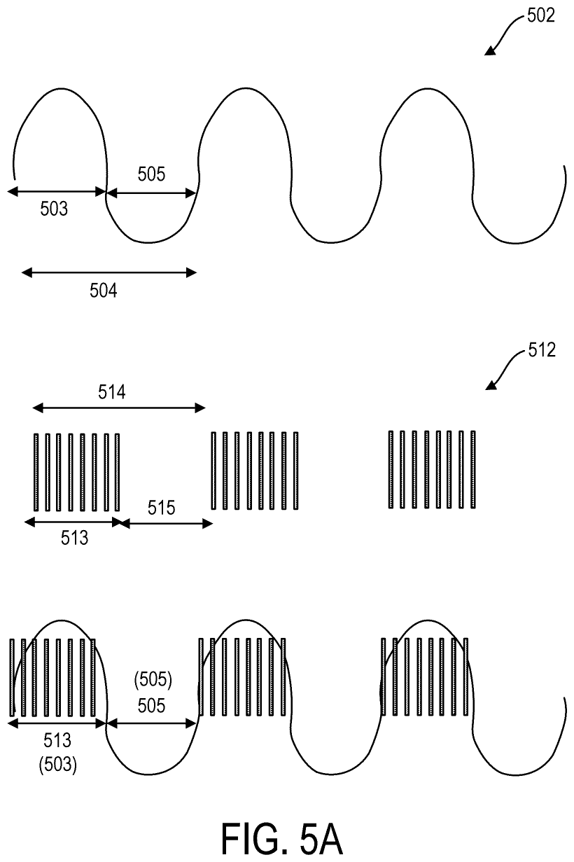

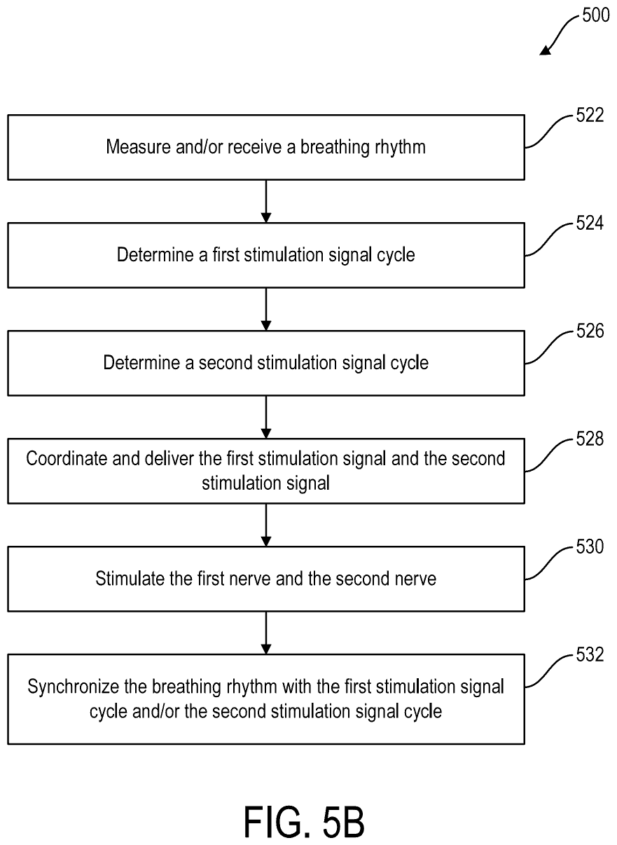

FIG. 5A is a schematic diagram illustrating neuromodulation entrainment with natural breathing rhythm, in accordance with embodiments of the present disclosure. The example respiratory waveform 502 includes a series of successively repeated respiratory cycles 504 each including an inspiratory phase 503 and an expiratory phase 505. In some embodiments, the example respiratory waveform 502 can be pre-measured or predetermined for a specific patient. The respiratory waveform 502 can be stored as a historical respiratory waveform in a data repository of a medical system described herein.

In some embodiments, a stimulation signal 512 can include a series of stimulation signal cycles 514 each including a stimulation period 513 and a non-stimulation period 515. In some embodiments, a stimulation signal cycle 514 of the stimulation signal 512 can be controlled or adjusted to have a duration substantially the same as the duration of a respiratory cycle of the historical respiratory waveform 502. For example, the difference between the duration of a stimulation signal cycle 514 of the stimulation signal 512 and the duration of a respiratory cycle 504 of the respiratory waveform 502 can be no great than 20%, no greater than 15%, or no greater than 10%. In some embodiments, the stimulation period 513 can have a duration substantially the same as the duration of the inspiratory phase 503 of the respiratory waveform 502. For example, the difference between the duration of the stimulation period 513 and the duration of the inspiratory phase 503 of the respiratory waveform 502 can be no great than 20%, no greater than 15%, or no greater than 10%. In some embodiments, the non-stimulation period 515 can have a duration substantially the same as the duration of the expiratory phase 505 of the respiratory waveform 502. For example, the difference between the duration of the non-stimulation period 515 and the duration of the expiratory phase 505 of the respiratory waveform 502 can be no great than 20%, no greater than 15%, or no greater than 10%.

In some embodiments, the medical system 200 does not require a sensor to detect the patient's respiratory waveform in real time. Instead, the controller 210 can access the historical respiratory waveform 502 stored in the data repository 212. The controller 210 can determine one or more stimulation parameters based at least in part on the historical respiratory waveform 502. For example, the controller 210 can determine the duration(s) of the stimulation signal 512 based at least in part on the respective duration(s) of the respiratory waveform 502. The controller 210 can send the determined stimulation parameters to the stimulation signal generator 206 to generate and deliver one or more stimulation signals to one or more of the corresponding implantable electrodes.

In some embodiments, the stimulation signal generator 206 generates a first stimulation signal to deliver to the first implantable electrode 202 to stimulate the first nerve and activate at least one muscle for an upper airway dilation for the patient. While not wanting to be bound by theory, it is believed that the duration of upper airway patency can follow the first stimulation cycle of the first stimulation signal, which in turn can make the patient's natural breathing rhythm follow and synchronize with the first stimulation cycle of the first stimulation signal, in other words, the entrainment of the patient's natural breathing rhythm with the first stimulation signal. For example, in the embodiment depicted in FIG. 5, the stimulation signal 512 can be the first stimulation signal. The stimulation signal cycle 513 of the first stimulation signal 512 is substantially synchronized with the respiratory cycle of the patient's respiratory waveform 502. The onset of the stimulation period 513 of the first stimulation signal 512 is substantially aligned with the onset of the inspiratory phase 503 of the patient's respiratory waveform 502.

In some embodiments, the stimulation signal generator 206 generates a second stimulation signal to deliver to the second implantable electrode 204 to stimulate the second nerve and activate at least one muscle for a caudal tracheal traction for an upper airway for the patient. While not wanting to be bound by theory, it is believed that the duration of upper airway patency can follow the second stimulation cycle of the second stimulation signal, which in turn can make the patient's natural breathing rhythm follow and synchronize with the second stimulation cycle of the second stimulation signal, in other words, the entrainment of the patient's natural breathing rhythm with the second stimulation signal. For example, in the embodiment depicted in FIG. 5, the stimulation signal 512 can be the second stimulation signal. The stimulation signal cycle 513 of the second stimulation signal 512 is substantially synchronized with the respiratory cycle of the patient's respiratory waveform 502. The onset of the stimulation period 513 of the second stimulation signal 512 is substantially aligned with the onset of the inspiratory phase 503 of the patient's respiratory waveform 502.

FIG. 5B is a flow diagram of a medical method 500 for managing obstructive sleep apnea of a patient through upper airway dual neurostimulation, in accordance with embodiments of the present disclosure.

It is to be understood that any stimulation signal generators and electrodes can be used in the method. Aspects of embodiments of the method may be performed, for example, by a medical system or a controller (e.g., the medical system 200 in FIG. 2, the controller 210 in FIG. 2). One or more steps or blocks of method are optional and/or can be modified by one or more steps of other embodiments described herein. Additionally, one or more steps of other embodiments described herein may be added to the method.

According to certain embodiments, at process 522, the patient's natural breathing rhythm, such as the respiratory waveform 502 of FIG. 5A, can be measured or received. It is to be understood that the patient's natural breathing rhythm can be pre-measured or predetermined for a specific patient. In some embodiments, the controller 210 can access to the data repository 212 to obtain a historical respiratory waveform for the specific patient.

According to certain embodiments, at process 524, a first stimulation signal cycle of the first stimulation signal can be determined based at least in part on the patient's respiratory waveform. In some examples, the first stimulation signal cycle of the first stimulation signal (e.g., the stimulation signal cycle 514 of the stimulation signal 512 of FIG. 5A) can be controlled or adjusted to have a duration substantially the same as the duration of a respiratory cycle of the historical respiratory waveform 502.

According to certain embodiments, at process 526, a second stimulation signal cycle of the second stimulation signal can be determined based at least in part on the patient's respiratory waveform and/or the first stimulation signal. In some examples, the second stimulation signal cycle of the second stimulation signal (e.g., the stimulation signal cycle 514 of the stimulation signal 512 of FIG. 5A) can be controlled or adjusted to have a duration substantially the same as the duration of a respiratory cycle of the historical respiratory waveform 502.

According to certain embodiments, at process 528, the first stimulation signal and the second stimulation signal can be coordinated to be delivered proximate to a first nerve and a second nerve, respectively.

According to certain embodiments, at process 530, the first stimulation signal is delivered to stimulate the first nerve, and the second stimulation signal is delivered to stimulate the second nerve. In some embodiments, the first stimulation signal is delivered to stimulate a first hypoglossal nerve to activate tongue protrusors (e.g., genioglossus), and avoid stimulating certain nerve branches which may activate suprahyoid muscles (e.g., geniohyoid) and avoid stimulating certain nerve branches which may activate retractor muscles of the tongue muscles (e.g., styloglossus and/or hyoglossus). In some embodiments, the second stimulation signal is delivered to stimulate the first ansa cervicalis nerve to activate infrahyoid muscles (e.g., sternothyroid and/or sternohyoid), and avoid stimulating certain nerve branches which may activate suprahyoid muscles (e.g., geniohyoid). In some embodiments, the second implantable electrode can be implanted to deliver the second stimulation signal to stimulate certain nerve branch(es) of the ansa cervicalis and activate sternothyroid and sternohyoid muscles simultaneously, while avoiding stimulating certain nerve branches which may activate suprahyoid muscles (e.g., geniohyoid).

According to certain embodiments, at process 532, the patient's breathing rhythm can be synchronized with the first stimulation signal cycle and/or the second stimulation signal cycle to achieve the entrainment of the patient's natural breathing rhythm with the dual neurostimulation by using the coordinated first and second stimulation signals.

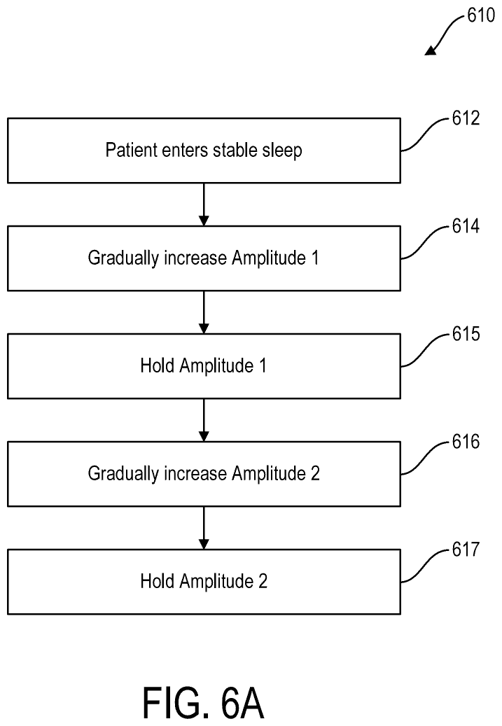

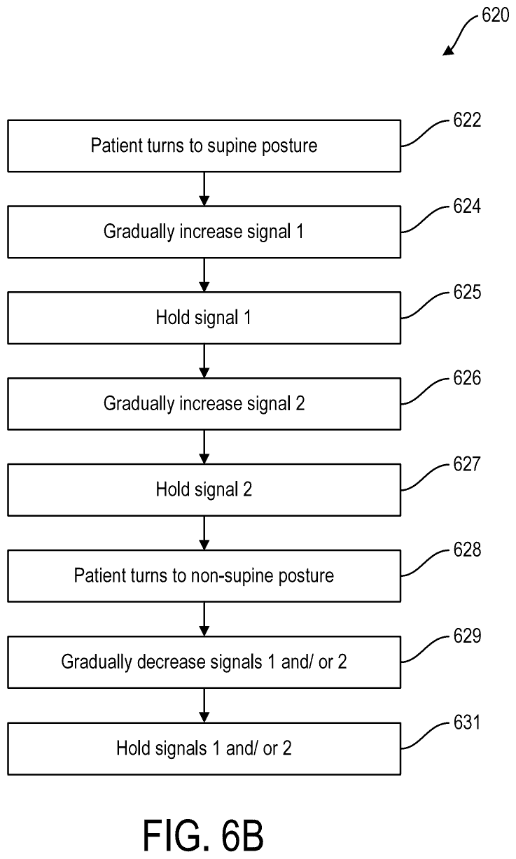

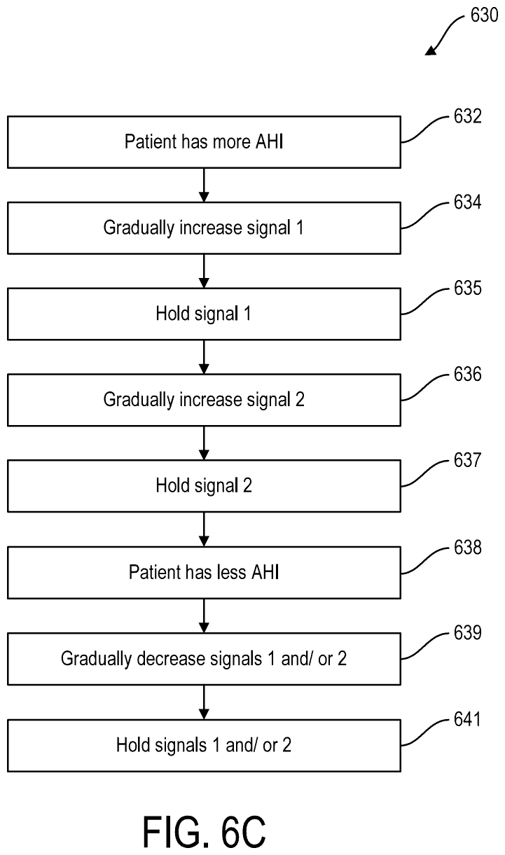

FIGS. 6A-C are flow diagrams illustrating various example titration methods to develop stimulation therapies or settings for managing obstructive sleep apnea (OSA) for a person, in accordance with some embodiments of the present disclosure. In some embodiments, the obtained stimulation therapies or settings can be provided to the controller 210 or the stimulation signal generator 206 as programmable settings to modulate one or more stimulation signals.

In a first titration method 610, as illustrated in FIG. 6A, when a patient is detected entering a stable sleep at block 612, the first stimulation signal is gradually increased from a lower level to a higher level at block 614 and held at the higher level for a first period at block 615. The lower level can be, for example, a lower pulse amplitude, a lower pulse width, a lower duty cycle, a lower pulse frequency, and the like. The higher level can be, for example, a higher pulse width, a higher pulse amplitude, a higher duty cycle, a higher pulse frequency, and the like. In some embodiments, the lower level can be a functional or sensational threshold level at which the patient starts to feel the stimulation. For example, the lower level can be a level at which the patient's tongue starts protruding through stimulation of the first stimulation signal at the functional (bulk movement of the tongue or hyoid and thyroid complex) or sensational (the lowest level of stimulation with patient perception of stimulation) threshold level. In some embodiments, the higher level can be, for example, a saturation level at which the patient's AHI starts to be stabilized. With the first stimulation signal being held at the saturation level, the second stimulation signal can be gradually increased from a lower level to a higher level at block 616 and held at the higher level for a second period at block 617. It is to be understood that the lower/higher level of the first stimulation signal can be different from the lower/higher level of the second stimulation signal, which may depend on the characteristics of the respective first and second nerves to be stimulated.

In a second titration method 620, as illustrated in FIG. 6B, when a patient is detected turning to a supine posture at block 622, the first stimulation signal is gradually increased from a lower level to a higher level at block 624 and held at the higher level for a first period at block 625. The lower level can be, for example, a lower pulse amplitude, a lower pulse width, a lower duty cycle, and the like. The higher level can be, for example, a higher pulse width, a higher pulse amplitude, a higher duty cycle, and the like. In some embodiments, the lower level can be a functional or sensational threshold level at which the patient starts to feel the stimulation. For example, the lower level can be a level at which the patient's tongue starts protruding through stimulation of the first stimulation signal at the functional or sensational threshold level. In some embodiments, the higher level can be, for example, a saturation level at which the patient's AHI starts to be stabilized. With the first stimulation signal being held at the saturation level, the second stimulation signal can be gradually increased from a lower level to a higher level at block 626 and held at the higher level for a second period at block 627. It is to be understood that the respective lower levels and higher levels for the first and second stimulation signals may be different. When the patient is detected turning to a non-supine posture at block 628, the first stimulation signal and/or the second stimulation signal can be gradually decreased from the respective higher levels to respective second lower levels at block 629 and held at the respective second lower levels for a third period at block 631. The second lower levels may be the same as or different from the previous lower levels.

In a third titration method 630, as illustrated in FIG. 6C, when a patient is detected having an increased AHI at block 632, the first stimulation signal is gradually increased from a lower level to a higher level at block 634 and held at the higher level for a first period at block 635. The lower level can be, for example, a lower pulse amplitude, a lower pulse width, a lower duty cycle, and the like. The higher level can be, for example, a higher pulse width, a higher pulse amplitude, a higher duty cycle, and the like. In some embodiments, the lower level can be a functional or sensational threshold level at which the patient starts to feel the stimulation. For example, the lower level can be a level at which the patient's tongue starts protruding through stimulation of the first stimulation signal at the functional or sensational threshold level. In some embodiments, the higher level can be, for example, a saturation level at which the patient's AHI starts to be stabilized. With the first stimulation signal being held at the saturation level, the second stimulation signal can be gradually increased from a lower level to a higher level at block 636 and held at the higher level for a second period at block 637. When a patient is detected having the AHI decreased to a predetermined range (e.g., 5 or less) at block 638, the first stimulation signal and/or the second stimulation signal can be gradually decreased from the respective higher levels to respective second lower levels at block 639 and held at the respective second levels for a fourth period at block 641.

According to certain embodiments, in any of the titration methods (e.g., the first, second or third titration method 610, 620, 630), the patient's AHI can be monitored by the sensors 214 of FIG. 2 in real time to adjust one or more first parameters (e.g., the first amplitude, the first period, and the like) of the first stimulation signal and one or more second parameters (e.g., the second amplitude, the second period, and the like) of the second stimulation signal to optimize the method and obtain an optimized or predicted therapy for managing obstructive sleep apnea (OSA) for the patient.

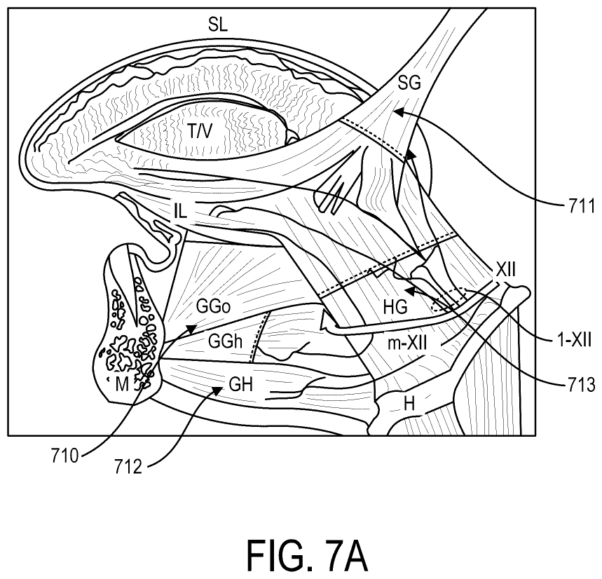

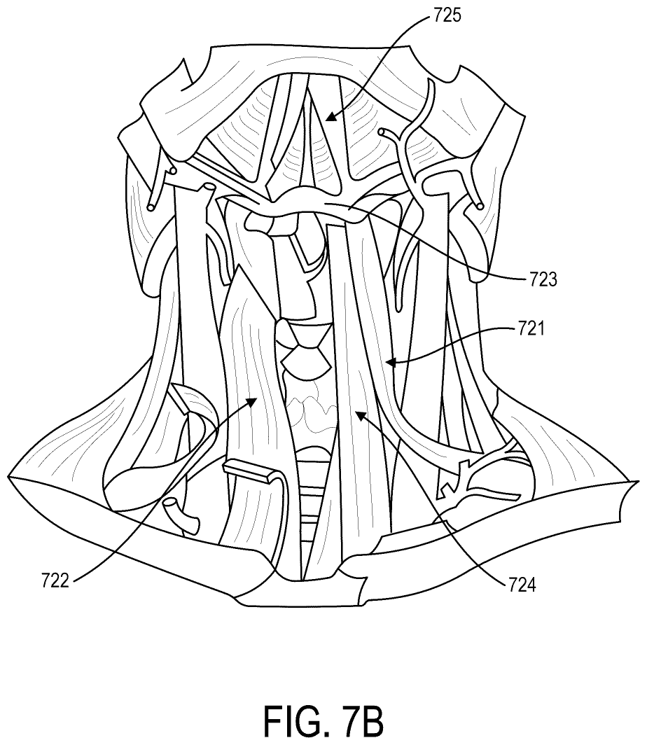

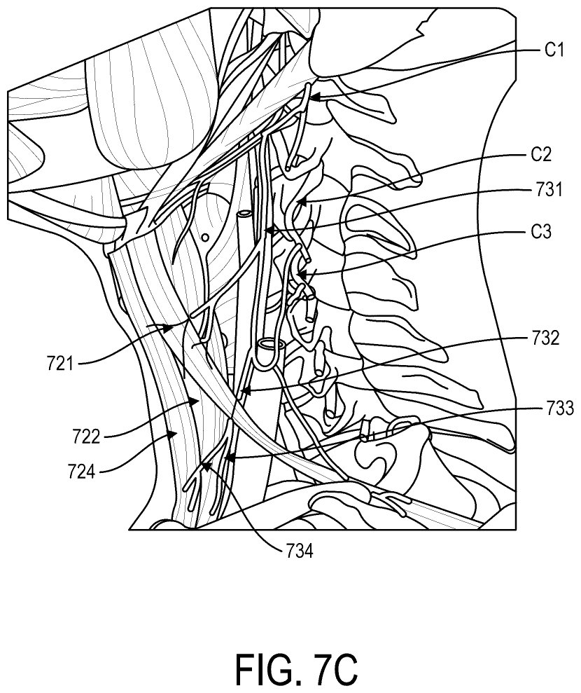

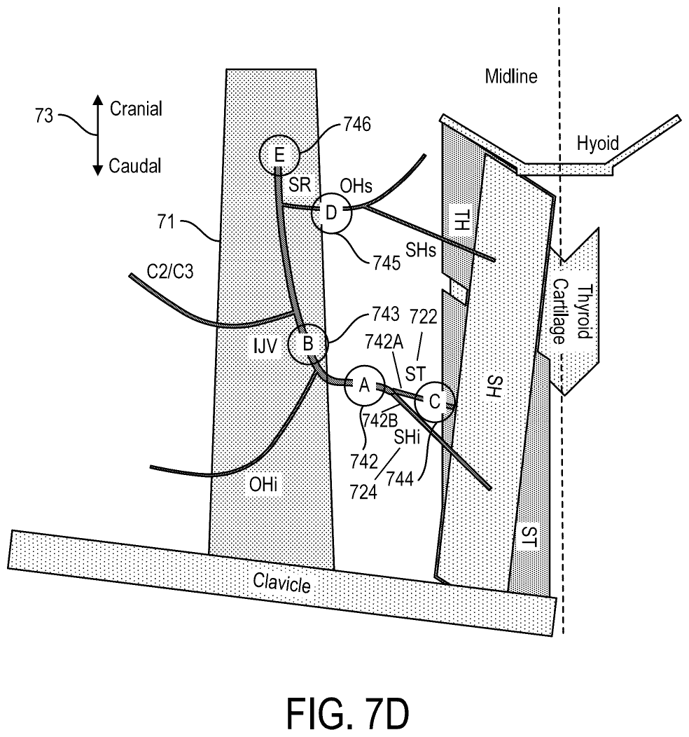

FIG. 7A illustrates a schematic diagram representing patient anatomy and target muscle location(s) for hypoglossal nerve stimulation, in accordance with embodiments of the present disclosure. In some embodiments, one or more first implantable electrodes deliver a first stimulation signal proximate to a first hypoglossal nerve to activate tongue protrusors (e.g., genioglossus 710). In some embodiments, an implantable electrode can be positioned to deliver the first stimulation signal proximate to one or more medial branches of the hypoglossal nerve (m-XII) to stimulate the one or more medial branches and activate one or more protrusion muscles of the tongue muscles including genioglossus 710. In some embodiments, the one or more first implantable electrodes deliver the first stimulation signal to avoid activating suprahyoid muscles (e.g., geniohyoid 712). In some embodiments, the one or more first implantable electrodes are positioned to avoid activating one or more retractor muscles of the tongue muscles including styloglossus 711 and hyoglossus 713.

FIG. 7B illustrates a schematic diagram representing patient anatomy and target muscle location(s) for ansa cervicalis stimulation, in accordance with embodiments of the present disclosure. In some embodiments, one or more second implantable electrodes deliver a second stimulation signal proximate to a first ansa cervicalis nerve to stimulate the first ansa cervicalis nerve to activate infrahyoid muscles including sternothyroid 722, sternohyoid 724, and omohyoid 721, which are below the hyoid 723. In some embodiments, the second implantable electrode can be implanted to deliver the second stimulation signal to stimulate certain nerve branch(es) of the ansa cervicalis and activate sternothyroid 722 and sternohyoid 724 simultaneously. In some embodiments, the one or more first implantable electrodes and second implantable electrodes are positioned to deliver the first and second stimulation signals to avoid activating suprahyoid muscles 725 (see also, e.g., geniohyoid 712 of FIG. 7A) which are above the hyoid 723. Activating the suprahyoid muscles 725 may counteract the effects of activating the infrahyoid muscles including sternothyroid 722 and sternohyoid 724 and it can be desirable to avoid activating the suprahyoid muscles 725 by properly positioning the first implantable electrodes.

It is to be understood that when the first stimulation signal is used alone (i.e., not working with the second stimulation signal at the same time) to activate tongue protrusors, it may be acceptable for the first stimulation signal to also activate the suprahyoid muscles (e.g., geniohyoid). When an upper airway dual neurostimulation including both hypoglossal nerve stimulation and ansa cervicalis stimulation is implemented as described herein, in some embodiments, it is desirable to avoid activating the suprahyoid muscles (e.g., geniohyoid) since such an activating may counteract the effects of the ansa cervicalis stimulation.

FIG. 7C illustrates a schematic diagram representing patient anatomy and target nerve/muscle location(s) for an upper airway dual neurostimulation including hypoglossal nerve stimulation and ansa cervicalis stimulation, in accordance with embodiments of the present disclosure. In some embodiments, the target or desired nerve/muscle location(s) for the upper airway dual neurostimulation can include, for example, one or more infrahyoid muscles (e.g., sternothyroid 722, sternohyoid 724, and omohyoid 721). FIG. 7C further illustrates the relevant locations such as a common trunk 731 to omohyoid, sternothyroid and sternohyoid, a common trunk 732 to sternothyroid and sternohyoid, a never trunk 733 to sternothyroid, a never trunk 734 to sternohyoid, a first cervical (C1) spinal nerve, a second cervical (C2) spinal nerve, and a third cervical (C3) spinal nerve. In some embodiments, the upper airway dual neurostimulation can avoid stimulating or activating the nerve/muscle location(s) including, for example, styloglossus, hyoglossus, and geniohyoid.

FIG. 7D illustrates a schematic diagram representing patient anatomy and target nerve/muscle location(s) for ansa cervicalis stimulation in an upper airway dual neurostimulation, in accordance with embodiments of the present disclosure. FIG. 7D illustrates the locations for various ansa cervicalis nerves including the superior root (SR), the omohyoid superior (OHs), the sternohyoid superior (SHs), the sternothyroid (ST), the sternohyoid inferior (SHi), and the omohyoid inferior (OHi). FIG. 7D also illustrates the locations for various muscles including the sternohyoid muscle (SH), the sternothyroid muscle (ST), and the thyrohyoid muscle (TH). The internal jugular vein (IJV) 71 and the cranial/caudal direction 73 are also shown. The target stimulation locations can include one or more of location A 742, location B 743, location C 744, location D 745, and location E 746. In some embodiments, a single implanted simulation lead (e.g., an electrode, an implantable electrode, and the like) can be located at or proximate to one of locations A to E 742, 743, 744, 745 and 746. For example, a single implanted simulation lead can be located at or proximate to location A 742 to deliver a stimulation signal. In some embodiments, the stimulation signal can be delivered at or proximate to location A 742 to stimulate two or more relevant nerve branch(es) of the ansa cervicalis simultaneously, for example, to activate sternothyroid (ST) 722 and sternohyoid (SHi) 724 simultaneously. In certain embodiments, a stimulation lead can be disposed (e.g., delivered to) at or proximate to a nerve segment connecting to two or more nerve branches. For example, the stimulation lead can be disposed at or proximate to the location 742 that is a nerve segment connecting to branch 742A and branch 742B. In some embodiments, the stimulation signal can be delivered at or proximate to a nerve segment connecting to two or more nerve branches.

In some embodiments, a first implanted simulation lead can be located (e.g., disposed) at or proximate to one of location A 742, location B 743 and location C 744, and a second implanted simulation lead can be located at or proximate to location E 746 or location D 745. In some embodiments, location A 742 and location B 743 can be superior to location C 744 due to the relatively closer distance to the internal jugular vein (IJV) 741 and/or the relatively larger nerve size (as indicated by the respective line thicknesses). In some embodiments, a first implanted simulation lead can be located at or proximate to location A 742 or B 743, and a second implanted simulation lead can be located at or proximate to location E 746.

In some embodiments, the controller 210 is configured to modulate the second stimulation signal between a lower stimulation level and a higher stimulation level to activate the one or more infrahyoid muscles within a strain range between a first strain value and a second strain value. In some embodiments, the strain can be measured by a displacement of the hyoid bone. The displacement can be an absolute displacement in a range, for example, from about 5 mm to about 20 mm, which can correspond to the first strain value and the second strain value, respectively. It is to be understood that the displacement or strain can be represented by a relative value, for example, a percentage. A lower level of the second stimulation signal corresponds to the first strain value, and a higher level of the second stimulation signal corresponds to the second strain value. An optimum stimulation level can be determined between the lower stimulation level and the higher stimulation level to provide an optimum strain to provide a caudal tracheal traction for an upper airway of the patient. The optimum strain level can prevent possible upper airway collapse or flow limitation. In some embodiments, the optimum stimulation level can be determined by using the measured strain levels (e.g., as indicated by the displacement of the hyoid bone) as feedback. For example, a series of stimulation levels can be applied, and the corresponding the displacements of the hyoid bone can be measured to determine the optimum stimulation level.

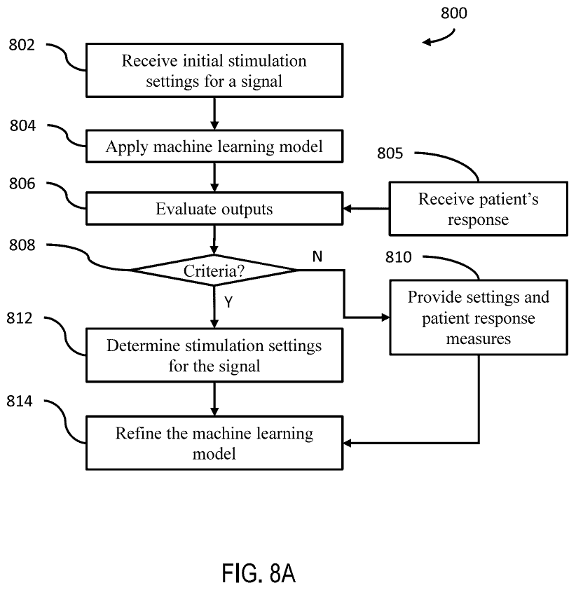

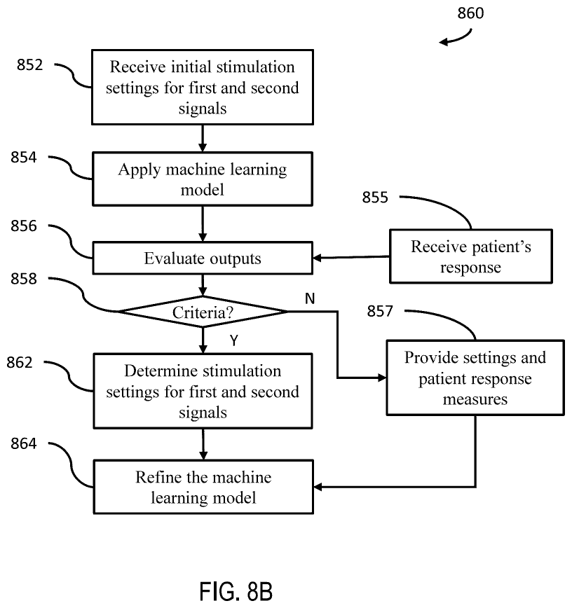

FIG. 8A is a flow diagram of a method 800 for determining stimulation settings for managing obstructive sleep apnea of a patient through upper airway neurostimulation, in accordance with embodiments of the present disclosure. According to some embodiments, stimulation settings are determined by using one or more computing models.

In certain embodiments, a model, also referred to as a computing model, includes a model to process data. A model includes, for example, an artificial intelligence (AI) model, a machine learning (ML) model, a deep learning (DL) model, an image processing model, an algorithm, a rule, other computing models, and/or a combination thereof. In some embodiments, a generative AI (artificial intelligence) model includes training data embedded in the model. In certain embodiments, a generative AI model is a type of AI model that can be used to produce various type of content, such as text, images, videos, audio, 3D (three-dimensional) data, 3D models, and/or the like. In some embodiments, a language model or a large language model (LLM), which is a type of generative AI models, includes content and training data embedded in the model.

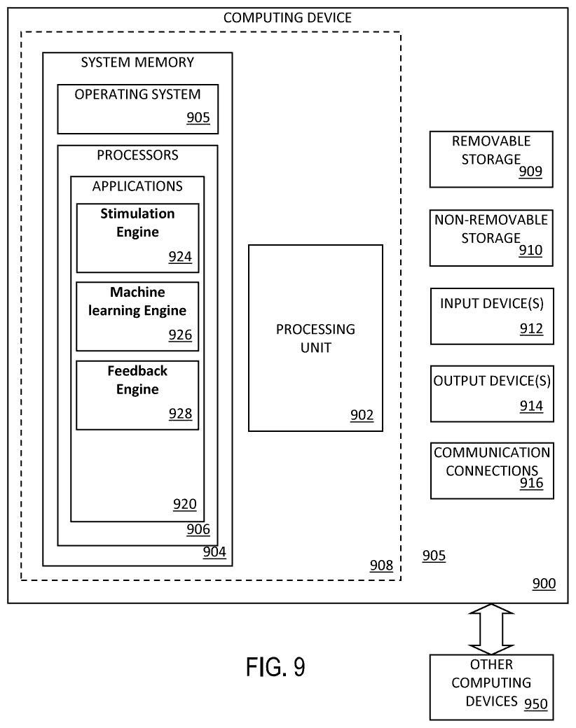

Aspects of embodiments of the method 800 can be performed, for example, by a medical system describe herein (e.g., the system 200 depicted in FIG. 2). According to some embodiments, the system (e.g., the system 200) includes one or more computing devices (e.g., the controller 210, the device 900 of FIG. 9) or servers which can be relied on to perform any of a variety of different tasks in the method 800. Further, some of the different tasks may be executed using automated systems. One or more steps or blocks of method 800 are optional and/or can be modified by one or more steps of other embodiments described herein. Depending upon the embodiment, the sequence of steps may be interchanged with others replaced. Additionally, one or more steps or blocks of other embodiments described herein may be added to the method 800.

According to certain embodiments, at process 802, the system (e.g., the system 200, the controller 210) receives a set of initial stimulation settings for a stimulation signal to be delivered by an electrode (e.g., the implantable electrode 202 or 204). In some embodiments, a first stimulation signal is generated based on the set of initial stimulation settings and delivered by a first implantable electrode. In some embodiments, a second stimulation signal is generated based on the set of initial stimulation settings and delivered by a second implantable electrode. The set of initial stimulation settings can include, for example, a start clock time, an end clock time, an amplitude, a frequency, a pulse width, a duty cycle, a ramped portion, and the like. According to some examples, when the first or second stimulation signal is applied to the patient in sleep, the patient's response can be measured in real time or collected after sleep. The measured or collected response can include, for example, the patient's feedback after sleep (e.g., patient's satisfaction level after sleep), a hypoxia burden level, an AHI value, the patient's daytime sleepiness level (e.g., as measured by Epworth sleepiness scale (ESS)), and the like.

According to certain embodiments, at process 804, the set of initial stimulation settings is provided to a trained machine learning model to generate outputs (e.g., predictions, classifications, and the like) associated with the patient's response. In some embodiments, a stimulation setting can include a stimulation parameter and a correspond value for the stimulation parameter. In some embodiments, the machine learning model can be a pre-trained machine learning model. For example, the machine learning model can be trained using historical stimulation settings and historical patient physiological parameters. In some embodiments, the set of initial stimulation settings can be manually set by a user. In some embodiments, the set of initial stimulation settings can be selected from historical stimulation settings. In some embodiments, the set of initial stimulation settings can be generated by applying a machine learning model with or without referencing to historical stimulation settings. In some embodiments, the machine learning model can include a supervised learning model using labeled data pairs as training data. For example, a labeled data pair can include first and second sets of initial stimulation settings with given parameters and the corresponding patient's response (e.g., a hypoxia burden level, AHI, ODI, RDI, a muscle strain value/level, a patient's satisfaction level after sleep, a patient's daytime sleepiness level, and the like). In some embodiments, the machine learning model can include an unsupervised learning model operating on unlabeled data. Suitable unsupervised learning model(s) can be applied to discover hidden patterns, structures, or relationships between the input of stimulation settings and the patient's corresponding response. It is to be understood that the machine learning model can be trained by any suitable methods other than supervised learning and unsupervised learning. In some embodiments, the machine learning model can be a pretrained model which can be fine-tuned for the specific tasks described herein.

According to certain embodiments, at process 805, the system (e.g., the system 200, the controller 210) receives one or more patient's responses (e.g., AHI, ODI, RDI, a muscle strain value/level, and the like) corresponding to the set of initial stimulation settings for a stimulation signal. In some embodiments, external sensors (e.g., the sensors 214) can detect one or more physiological information of the patient as the corresponding patient's response.

According to certain embodiments, at process 806, the outputs of the machine learning model can be evaluated by, for example, comparing to the measured or collected patient's response at process 805. In some embodiments, evaluation metrics can be used to assess the model's performance using historical data which may or may not be used for training the model. According to some examples, the outputs of the machine learning model can include one or more parameters indicating one or more predicted patient's response such as, for example, physiological information of the patient including AHI, ODI, RDI, a muscle strain value/level, and the like.

According to certain embodiments, at process 808, the system (e.g., the system 200, the controller 210) can determine whether the outputs of the machine learning model satisfy predetermined criteria based on the results of the evaluation at process 806. In some embodiments, the predetermined criteria can be one or more threshold levels to evaluate the difference between the outputs of the machine learning model and the measured or collected patient's response. For example, a threshold to evaluate the difference between the predicted AHI value and the measured or collected AHI value can be 3 or less. When the outputs of the machine learning model satisfy predetermined criteria, the method 800 proceeds to process 812. When the outputs of the machine learning model do not satisfy the predetermined criteria, the method 800 proceeds to process 810.

According to certain embodiments, at process 810, when the outputs of the machine learning model do not satisfy the predetermined criteria, the controller 210 can provide first feedback to continuously train/retune/retrain/refine the machine learning model at process 814. The first feedback can include settings and patient response measures such as, for example, data pairs including first and second sets of initial stimulation settings with given parameters and the corresponding patient's response. For example, the outputs of applying the machine learning can predict a strain value or range to activate the one or more infrahyoid muscles corresponding to an input of initial setting for the second stimulation signal. The difference between the predicted strain value or range can be compared to the measured strain (e.g., as indicated by a displacement of the hyoid bone). When the difference is greater than a predetermined upper threshold or lower than a predetermined lower threshold, first feedback can be provided to further tune the machine learning model at process 814 by, for example, adjusting parameters of the model. The tuned model can determine an optimum stimulation level between a lower stimulation level and a higher stimulation level to provide an optimum strain for a caudal tracheal traction for an upper airway of the patient. In some embodiments, a series of initial stimulation settings (e.g., stimulation levels) and/or a combination of initial stimulation settings can be applied as inputs for the machine learning model, and the corresponding outputs can be evaluated with respect to the predetermined criteria to provide feedback to tune the machine learning model.

According to certain embodiments, at process 812, when the outputs of the machine learning model satisfy predetermined criteria, the outputs of the machine learning model can be processed to determine stimulation settings with predicted parameters. In some embodiments, the outputs of applying the machine learning model can be evaluated with respect to the predetermined criteria to generate a set of predicted or optimized stimulation settings for the stimulation signal. In some embodiments, the determined stimulation settings can be provided as second feedback to retune/retrain/refine the machine learning model at process 814. For example, a set of predicted or optimized stimulation settings can be determined for the stimulation signal to be delivered by the electrode.

FIG. 8B is a flow diagram of a method 860 for determining stimulation settings for managing obstructive sleep apnea of a patient through upper airway neurostimulation, in accordance with embodiments of the present disclosure. According to some embodiments, stimulation settings are determined by using one or more computing models.

In certain embodiments, a model, also referred to as a computing model, includes a model to process data. A model includes, for example, an artificial intelligence (AI) model, a machine learning (ML) model, a deep learning (DL) model, an image processing model, an algorithm, a rule, other computing models, and/or a combination thereof. In some embodiments, a generative AI (artificial intelligence) model includes training data embedded in the model. In certain embodiments, a generative AI model is a type of AI model that can be used to produce various type of content, such as text, images, videos, audio, 3D (three-dimensional) data, 3D models, and/or the like. In some embodiments, a language model or a large language model (LLM), which is a type of generative AI models, includes content and training data embedded in the model.

Aspects of embodiments of the method 860 can be performed, for example, by a medical system describe herein (e.g., the system 200 depicted in FIG. 2). According to some embodiments, the system (e.g., the system 200) includes one or more computing devices (e.g., the controller 210, the device 900 of FIG. 9) or servers which can be relied on to perform any of a variety of different tasks in the method 860. Further, some of the different tasks may be executed using automated systems. One or more steps or blocks of method 860 are optional and/or can be modified by one or more steps of other embodiments described herein. Depending upon the embodiment, the sequence of steps may be interchanged with others replaced. Additionally, one or more steps or blocks of other embodiments described herein may be added to the method 860.

According to certain embodiments, at process 852, the system (e.g., the system 200, the controller 210) receives a first set of initial stimulation settings and a second set of initial stimulation settings. The first set of initial stimulation settings can be provided to a first stimulation signal to be delivered by a first electrode (e.g., the first implantable electrode 202). The second set of initial stimulation settings can be provided to a second stimulation signal to be delivered by a second electrode (e.g., the second implantable electrode 204). Each stimulation setting includes a stimulation parameter and a correspond value for the stimulation parameter. The first and second sets of initial stimulation settings are provided such that the first stimulation signal and the second stimulation signal are initially coordinated. According to some examples, when the coordinated first and second stimulation signals are applied to the patient in sleep, the patient's response can be measured in real time or collected after sleep. The measured or collected response can include, for example, the patient's feedback after sleep (e.g., patient's satisfaction level after sleep), a hypoxia burden level, an AHI value, the patient's daytime sleepiness level (e.g., as measured by Epworth sleepiness scale (ESS)), and the like.

According to certain embodiments, at process 854, the first set of initial stimulation settings and the second set of initial stimulation settings are provided to a machine learning model to generate outputs (e.g., predictions, classifications, and the like) associated with the patient's response. In some embodiments, a stimulation setting can include a stimulation parameter and a correspond value for the stimulation parameter. In some embodiments, the machine learning model can be a pre-trained machine learning model. For example, the machine learning model can be trained using historical stimulation settings and historical patient physiological parameters. In some embodiments, the machine learning model can include a supervised learning model using labeled data pairs as training data. For example, a labeled data pair can include first and second sets of initial stimulation settings with given parameters and the corresponding patient's response (e.g., a hypoxia burden level, AHI, ODI, RDI, a muscle strain value/level, the patient's satisfaction level after sleep, the patient's daytime sleepiness level, and the like). In some embodiments, the machine learning model can include an unsupervised learning model operating on unlabeled data. Suitable unsupervised learning model(s) can be applied to discover hidden patterns, structures, or relationships between the input of first and/or second stimulation settings and the patient's response. It is to be understood that the machine learning model can be trained by any suitable methods other than supervised learning and unsupervised learning. In some embodiments, the machine learning model can be a pretrained model which can be fine-tuned for the specific tasks described herein.

According to certain embodiments, at process 855, the system (e.g., the system 200, the controller 210) receives one or more patient's responses (e.g., AHI, ODI, RDI, a muscle strain value/level, and the like) corresponding to the first set of initial stimulation settings and the second set of initial stimulation settings. In some embodiments, external sensors (e.g., the sensors 214) can detect one or more physiological information of the patient as the corresponding patient's response.

According to certain embodiments, at process 856, the outputs of the machine learning model can be evaluated by, for example, comparing to the measured or collected patient's response at process 855. In some embodiments, evaluation metrics can be used to assess the model's performance using historical data which may or may not be used for training the model. According to some examples, the outputs of the machine learning model can include one or more parameters indicating one or more predicted patient's response such as, for example, physiological information of the patient including AHI, ODI, RDI, a muscle strain value/level, and the like.

According to certain embodiments, at process 858, the system (e.g., the system 200, the controller 210) can determine whether the outputs of the machine learning model satisfy predetermined criteria based on the results of the evaluation at process 806. In some embodiments, the predetermined criteria can be one or more threshold levels to evaluate the difference between the outputs of the machine learning model and the measured or collected patient's response. For example, a threshold to evaluate the difference between the predicted AHI value and the measured or collected AHI value can be 3 or less. When the outputs of the machine learning model satisfy predetermined criteria, the method 860 proceeds to process 862. When the outputs of the machine learning model do not satisfy the predetermined criteria, the method 860 proceeds to process 857.

According to certain embodiments, at process 857, when the outputs of the machine learning model do not satisfy the predetermined criteria, the controller 210 can provide first feedback to retune/retrain/refine the machine learning model at process 814. The first feedback can include settings and patient response measures such as, for example, data pairs including first and second sets of initial stimulation settings with given parameters and the corresponding patient's response. For example, the outputs of applying the machine learning can predict a strain value or range to activate the one or more infrahyoid muscles corresponding to an input of initial setting for the second stimulation signal. The difference between the predicted strain value or range can be compared to the measured strain (e.g., as indicated by a displacement of the hyoid bone). When the difference is greater than a predetermined upper threshold or lower than a predetermined lower threshold, first feedback can be provided to further train the machine learning model at process 864 by, for example, adjusting parameters of the model. The tuned model can determine an optimum stimulation level between a lower stimulation level and a higher stimulation level to provide an optimum strain for a caudal tracheal traction for an upper airway of the patient. In some embodiments, a series of initial stimulation settings (e.g., stimulation levels) and/or a combination of initial stimulation settings can be applied as inputs for the machine learning model, and the corresponding outputs can be evaluated with respect to the predetermined criteria to provide feedback to tune the machine learning model.

According to certain embodiments, at process 862, when the outputs of the machine learning model satisfy predetermined criteria, the outputs of the machine learning model can be processed to determine stimulation settings with predicted parameters. In some embodiments, the outputs of applying the machine learning model can be evaluated with respect to the predetermined criteria to generate a first set of predicted or optimized stimulation settings for the first stimulation signal and a second set of predicted or optimized stimulation settings for the second stimulation signal. The first and second sets of stimulation settings are provided such that the first and second stimulation signals are coordinated in a way which may be different from the initial coordination. For example, the initial coordination can be in a first mode (e.g., the first mode in FIG. 4A), and the determined coordination can be in a second mode ((e.g., the second mode in FIG. 4B) different from the first mode. In some embodiments, the determined stimulation settings can be provided as second feedback to retune/retrain/refine the machine learning model at process 864. For example, a first set of predicted or optimized stimulation settings can be determined for the first stimulation signal, and a second set of predicted or optimized stimulation settings can be determined for the second stimulation signal to coordinate with the first stimulation signal.

FIG. 9 is a simplified block diagrams of a computing device 900, with which aspects of the present disclosure may be practiced. The computing device components described below may be suitable for the computing devices described above, including the controller 210 in FIG. 2 and any computing device and/or server functionally connected to the controller 210. The computing device 900 may include at least one processing unit 902 and a system memory 904. Depending on the configuration and type of computing device, the system memory 904 may include, for example, volatile storage (e.g., random access memory), non-volatile storage (e.g., read-only memory), flash memory, or any combination of such memories.

The system memory 904 may include an operating system 905 and one or more program modules 906 suitable for running software application 920, such as one or more components supported by the systems described herein. As examples, system memory 904 may store a stimulation engine or component 924, a machine learning engine or component 926, and/or a feedback engine or component 928. The operating system 905, for example, may be suitable for controlling the operation of the computing device 900.

In some embodiments, the stimulation engine or component 924 can determine stimulation settings for managing obstructive sleep apnea of a patient through upper airway neurostimulation.

In some embodiments, the machine learning engine or component 926 can apply a model (e.g., a generative AI model) to various stimulation settings. For example, the machine learning engine or component 926 can apply a trained machine learning model to a first set of initial stimulation settings and/or a second set of initial stimulation settings to generate outputs (e.g., predictions, classifications, and the like) associated with the patient's response.

In some embodiments, the feedback engine or component 928 can evaluate the outputs of the machine learning model to provide feedback and improve the machine learning model. For example, when the outputs of the machine learning model do not satisfy the predetermined criteria, the feedback engine or component 928 can provide first feedback to further tune the machine learning model. When the outputs of the machine learning model satisfy the predetermined criteria, the feedback engine or component 928 can provide second feedback to further tune the machine learning model. In some embodiments, the determined stimulation settings can be provided as the second feedback.

A basic configuration is illustrated in FIG. 9 by those components within a dashed line 908. The computing device 900 may have additional features or functionality. For example, the computing device 900 may also include additional data storage devices (removable and/or non-removable) such as, for example, magnetic disks, optical disks, or tape. Such additional storage is illustrated in FIG. 9 by a removable storage device 909 and a non-removable storage device 910.

As stated above, a number of program modules and data files may be stored in the system memory 904. While executing on the processing unit 902, the program modules 906 (e.g., application 920) may perform processes including, but not limited to, the aspects, as described herein. Other program modules that may be used in accordance with aspects of the present disclosure may include electronic mail and contacts applications, word processing applications, spreadsheet applications, database applications, slide presentation applications, drawing or computer-aided application programs, and the like.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 9 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 900 on the single integrated circuit (chip). Some aspects of the disclosure may also be practiced using other technologies capable of performing logical operations such as, for example, AND, OR, and NOT, including but not limited to mechanical, optical, fluidic, and quantum technologies. In addition, some aspects of the disclosure may be practiced within a general purpose computer or in any other circuits or systems.

The computing device 900 may also have one or more input device(s) 912 such as a keyboard, a mouse, a pen, a sound or voice input device, a touch or swipe input device, and the like. The output device(s) 914 such as a display, speakers, a printer, etc. may also be included. The aforementioned devices are examples and others may be used. The computing device 900 may include one or more communication connections 916 allowing communications with other computing devices 950. Examples of suitable communication connections 916 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 904, the removable storage device 909, and the non-removable storage device 910 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 900. Any such computer storage media may be part of the computing device 900. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

According to some embodiments of the present disclosure, a system for managing obstructive sleep apnea for a person is provided. The system includes an implantable electrode configured to deliver a stimulation signal proximate to a nerve of the person to stimulate the nerve and activate at least one muscle associated with an airway of the person; a stimulation signal generator configured to deliver the stimulation signal to the implantable electrode, wherein the stimulation signal includes a series of stimulation cycles each including a stimulation period and a non-stimulation period; and a controller functionally connected to the stimulation signal generator to control operation of the stimulation signal generator, the controller configured to: generate a set of predicted stimulation settings for the stimulation signal using a trained machine learning model; and provide the set of predicted stimulation settings to the stimulation signal generator.

In certain embodiments, the implantable electrode is a first implantable electrode. The stimulation signal is a first stimulation signal to activate at least one first muscle for an upper airway dilation of the person. The system further includes a second implantable electrode configured to deliver a second stimulation signal proximate to a second nerve to stimulate the second nerve and activate at least one second muscle for a caudal tracheal traction for an upper airway of the person. The controller is further configured to generate a second set of predicted stimulation settings for the second stimulation signal using the trained machine learning model; provide the second set of predicted stimulation settings to the stimulation signal generator; and coordinate the delivery of the first stimulation signal with the delivery of the second stimulation signal.

In certain embodiments, the implantable electrode is a first implantable electrode. The stimulation signal is a first stimulation signal to activate at least one first muscle for a caudal tracheal traction for an upper airway of the person. The system further comprises a second implantable electrode configured to deliver a second stimulation signal proximate to a second nerve of the person to stimulate the second nerve and activate at least one second muscle for an upper airway dilation of the person. The controller is further configured to generate a second set of predicted stimulation settings for the second stimulation signal using the trained machine learning model; provide the second set of predicted stimulation settings to the stimulation signal generator; and coordinate the delivery of the first stimulation signal with the delivery of the second stimulation signal.

According to certain embodiments of the present disclosure, a method for managing obstructive sleep apnea for a person is provided. The method includes providing an implantable electrode configured to deliver a stimulation signal proximate to a nerve to stimulate the nerve and activate at least one muscle associated with an air way of the person; generating a set of predicted stimulation settings for the stimulation signal using a trained machine learning model; providing the set of predicted stimulation settings to a stimulation signal generator; and delivering the stimulation signal to the implantable electrode. The stimulation signal has a series of stimulation cycles each including a stimulation period and a non-stimulation period.

In certain embodiments, the implantable electrode is a first implantable electrode. The stimulation signal is a first stimulation signal to activate at least one first muscle for an upper airway dilation of the person. The method further includes providing a second implantable electrode configured to deliver a second stimulation signal proximate to a second nerve to stimulate the second nerve and activate at least one second muscle for a caudal tracheal traction for an upper airway of the person; and generating a second set of predicted stimulation settings for the second stimulation signal using the trained machine learning model; providing the second set of predicted stimulation settings to the stimulation signal generator; and coordinating the delivery of the first stimulation signal with the delivery of the second stimulation signal.

In certain embodiments, the implantable electrode is a first implantable electrode to a first stimulation signal proximate to a first nerve of the person to stimulate the first nerve and activate at least one first muscle for a caudal tracheal traction for an upper airway of the person. The method further includes providing a second implantable electrode configured to deliver a second stimulation signal proximate to a second nerve of the person to stimulate the second nerve and activate at least one second muscle for an upper airway dilation of the person; generating a second set of predicted stimulation settings for the second stimulation signal using the trained machine learning model; providing the second set of predicted stimulation settings to the stimulation signal generator; and coordinating the delivery of the first stimulation signal with the delivery of the second stimulation signal.

According to some embodiments of the present disclosure, a system for managing obstructive sleep apnea for a person is provided. The system includes a first implantable electrode configured to deliver a first stimulation signal proximate to a first nerve of the person to stimulate the first nerve and activate at least one first muscle for an upper airway dilation of the person; and a second implantable electrode configured to deliver a second stimulation signal proximate to a second nerve to stimulate the second nerve and activate at least one second muscle for a caudal tracheal traction for an upper airway of the person. A stimulation signal generator is configured to deliver the first stimulation signal to the first implantable electrode, and deliver the second stimulation signal to the second implantable electrode. The first stimulation signal includes a series of first stimulation cycles each including a first stimulation period and a first non-stimulation period. The second stimulation signal includes a series of second stimulation cycles each including a second stimulation period and a second non-stimulation period. The delivery of the first stimulation signal is coordinated with the delivery of the second stimulation signal. A controller is functionally connected to the stimulation signal generator to control operation of the stimulation signal generator, the controller configured to apply a trained machine learning model to a first set of initial stimulation settings for the first stimulation signal and a second set of initial stimulation settings for the second stimulation signal to generate a first set of predicted stimulation settings for the first stimulation signal and a second set of predicted stimulation settings for the second stimulation signal.

In certain embodiments, the trained machine learning model is trained using historical stimulation settings and historical patient physiological parameters.

In certain embodiments, each stimulation setting includes a stimulation parameter and a correspond value for the stimulation parameter.

In certain embodiments, the one or more stimulation parameters include one or more of an amplitude, a frequency, a pulse width, a rate of amplitude change, and a duty cycle.

In certain embodiments, the system further includes one or more sensors to detect one or more physiological parameters including an apnea-hypopnea index (AHI), a posture change, a sleep stage, and a time of day.

In certain embodiments, the controller is configured to evaluate the first set of predicted stimulation settings and the second set of predicted stimulation settings based at least in part on the one or more physiological parameters.

In certain embodiments, the first implantable electrode is configured to deliver the first stimulation signal proximate to a hypoglossal nerve to stimulate the hypoglossal nerve and activate at least one tongue muscle.

In certain embodiments, the first implantable electrode is positioned to deliver the first stimulation signal proximate to one or more medial branches of the hypoglossal nerve (m-XII) to stimulate the one or more medial branches and activate one or more protrusion muscles of the at least one tongue muscle including genioglossus.

In certain embodiments, the second implantable electrode is configured to deliver the second stimulation signal proximate to an ansa cervicalis nerve to stimulate the ansa cervicalis nerve and activate one or more infrahyoid muscles.

In certain embodiments, the controller is configured to modulate the second stimulation signal to activate the one or more infrahyoid muscles within the strain range between the first strain value and the second strain value, wherein a first amplitude level of the second stimulation signal corresponds to the first strain value, and a second amplitude level of the second stimulation signal corresponds to the second strain value.

In certain embodiments, the controller is configured to control the operation of the stimulation signal generator in a first mode, wherein the stimulation signal generator is configured to synchronize the first stimulation periods and the first non-stimulation periods of the first stimulation signal with the second stimulation periods and the second non-stimulation periods of the second stimulation signal, respectively.

In certain embodiments, the controller is configured to control the operation of the stimulation signal generator in a second mode, wherein a first duration of the first stimulation period is greater than a second duration of the second stimulation period.

In certain embodiments, in the second mode, the first duration of the first stimulation period is two or more times greater than the second duration of the second stimulation period.

In certain embodiments, the stimulation signal generator is configured to coordinate the delivery of the first stimulation signal and the delivery of the second stimulation signal based at least in part on a historical respiratory waveform.

In certain embodiments, the stimulation signal generator comprises an internal timer to provide timing to coordinate the series of first stimulation cycles and the series of second stimulation cycles, independent of a respiratory status of the person.

In certain embodiments, the controller further comprises a patient controller to control operation of the stimulation signal generator, including to turn on or turn off the stimulation signal generator.

In certain embodiments, the system further includes a third implantable electrode configured to deliver a third stimulation signal proximate to a third nerve to stimulate the third nerve.

According to certain embodiments of the present disclosure, a method for managing obstructive sleep apnea for a person is provided. The method includes providing a first implantable electrode configured to deliver a first stimulation signal proximate to a first nerve to stimulate the first nerve and activate at least one first muscle for upper airway dilation; providing a second implantable electrode configured to deliver a second stimulation signal proximate to a second nerve to stimulate the second nerve and activate at least one second muscle for a caudal tracheal traction for an upper airway of the person; applying a trained machine learning model to a first set of initial stimulation settings for the first stimulation signal and a second set of initial stimulation settings for the second stimulation signal to generate a first set of predicted stimulation settings for the first stimulation signal and a second set of predicted stimulation settings for the second stimulation signal; delivering the first stimulation signal to the first implantable electrode; and delivering the second stimulation signal to the second implantable electrode. The first stimulation signal and the second stimulation signal are coordinated. The first stimulation signal has a series of first stimulation cycles each including a first stimulation period and a first non-stimulation period, and the second stimulation signal has a series of second stimulation cycles each including a second stimulation period and a second non-stimulation period.

In certain embodiments, the method further includes training a machine learning model using historical stimulation settings and historical patient physiological parameters to obtain the trained machine learning model.

In certain embodiments, each stimulation setting includes a stimulation parameter and a correspond value for the stimulation parameter, and one or more first stimulation parameters and one or more second stimulation parameters each include one or more of an amplitude, a frequency, a pulse width, a rate of amplitude change, and a duty cycle.

In certain embodiments, the method further includes detecting one or more physiological parameters including an apnea-hypopnea index (AHI), a posture change, a sleep stage, and a time of day.

In certain embodiments, the method further includes evaluating the first set of predicted stimulation settings and the second set of predicted stimulation settings based at least in part on the one or more physiological parameters.

In certain embodiments, the first implantable electrode is configured to deliver the first stimulation signal proximate to a hypoglossal nerve to stimulate the hypoglossal nerve and activate at least one tongue muscle.

In certain embodiments, delivering the first stimulation signal further includes delivering the first stimulation signal proximate to one or more medial branches of the hypoglossal nerve (m-XII) to stimulate the one or more medial branches and activate one or more protrusion muscles of the at least one tongue muscle including genioglossus.

In certain embodiments, the second implantable electrode is configured to deliver a second stimulation signal proximate to an ansa cervicalis nerve to stimulate the ansa cervicalis nerve and activate one or more infrahyoid muscles including sternothyroid and sternohyoid.

In certain embodiments, the method further includes modulating the second stimulation signal to activate the one or more infrahyoid muscles within the strain range between the first strain value and the second strain value, wherein a first amplitude level of the second stimulation signal corresponds to the first strain value, and a second amplitude level of the second stimulation signal corresponds to the second strain value.

In certain embodiments, the method further includes controlling operation of a stimulation signal generator in a first mode, wherein the stimulation signal generator is configured to synchronize the first stimulation periods and the first non-stimulation periods of the first stimulation signal with the second stimulation periods and the second non-stimulation periods of the second stimulation signal, respectively.

In certain embodiments, the method further includes controlling operation of a stimulation signal generator in a second mode, wherein a first duration of the first stimulation period is two or more times greater than a second duration of the second stimulation period.

In certain embodiments, the method further includes coordinating the delivery of the first stimulation signal and the delivery of the second stimulation signal based at least in part on a historical respiratory waveform of the person.

In certain embodiments, the method further includes synchronizing the series of first stimulation cycles of the first stimulation signal with a current respiratory waveform of the person.

In certain embodiments, the method further includes synchronizing the series of second stimulation cycles of the second stimulation signal with a current respiratory waveform of the person.

In certain embodiments, the method further includes providing timing to coordinate the series of first stimulation cycles and the series of second stimulation cycles by using an internal timer, independent of a respiratory status of the person.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A system for managing obstructive sleep apnea for a person, the system comprising:
an implantable electrode configured to deliver a stimulation signal proximate to a nerve of the person to stimulate the nerve and activate at least one muscle associated with an airway of the person;
a stimulation signal generator configured to deliver the stimulation signal to the implantable electrode, wherein the stimulation signal includes a series of stimulation cycles each including a stimulation period and a non-stimulation period; and
a controller functionally connected to the stimulation signal generator to control operation of the stimulation signal generator, the controller configured to:
generate a set of predicted stimulation settings for the stimulation signal using a trained machine learning model by at least:
generating a duration of stimulation cycle in the set of predicted stimulation settings by applying the trained machine learning model to a historical respiratory waveform of the person independent of a real-time respiratory waveform of the person; and
provide the set of predicted stimulation settings to the stimulation signal generator.

2. The system of claim 1, wherein the trained machine learning model is trained using historical stimulation settings and historical patient physiological parameters.

3. The system of claim 1, wherein each stimulation setting of the set of predicted stimulation settings includes a stimulation parameter and a corresponding value for the stimulation parameter.

4. The system of claim 3, wherein the stimulation parameter includes one or more of an amplitude, a frequency, a pulse width, a rate of amplitude change, and a duty cycle.

5. The system of claim 1, further comprising one or more sensors to detect one or more physiological parameters including an apnea-hypopnea index (AHI), a posture change, a sleep stage, a sleepiness measure, a hypoxia burden level, a patient sleep quality measure, and a time of day.

6. The system of claim 5, wherein the controller is configured to evaluate the set of predicted stimulation settings based at least in part on the one or more physiological parameters.

7. The system of claim 1, wherein the implantable electrode is a first implantable electrode, wherein the stimulation signal is a first stimulation signal to activate at least one first muscle for an upper airway dilation of the person;
wherein the system further comprises a second implantable electrode configured to deliver a second stimulation signal proximate to a second nerve to stimulate the second nerve and activate at least one second muscle for a caudal tracheal traction for an upper airway of the person;
wherein the controller is further configured to:
generate a second set of predicted stimulation settings for the second stimulation signal using the trained machine learning model;
provide the second set of predicted stimulation settings to the stimulation signal generator; and
coordinate the delivery of the first stimulation signal with the delivery of the second stimulation signal.

8. The system of claim 1, wherein the implantable electrode is a first implantable electrode, wherein the stimulation signal is a first stimulation signal to activate at least one first muscle for a caudal tracheal traction for an upper airway of the person;
wherein the system further comprises a second implantable electrode configured to deliver a second stimulation signal proximate to a second nerve of the person to stimulate the second nerve and activate at least one second muscle for an upper airway dilation of the person;
wherein the controller is further configured to:
generate a second set of predicted stimulation settings for the second stimulation signal using the trained machine learning model;
provide the second set of predicted stimulation settings to the stimulation signal generator; and
coordinate the delivery of the first stimulation signal with the delivery of the second stimulation signal.

9. The system of claim 1, wherein the implantable electrode is configured to deliver the stimulation signal proximate to a hypoglossal nerve to stimulate the hypoglossal nerve and activate at least one tongue muscle.

10. The system of claim 1, wherein the implantable electrode is configured to deliver the stimulation signal proximate to an ansa cervicalis nerve to stimulate the ansa cervicalis nerve and activate one or more infrahyoid muscles.

11. The system of claim 10, wherein the stimulation signal is further configured to activate sternothyroid and sternohyoid simultaneously.

12. A method for managing obstructive sleep apnea for a person, the method comprising:
providing an implantable electrode configured to deliver a stimulation signal proximate to a nerve to stimulate the nerve and activate at least one muscle associated with an air way of the person;
generating a set of predicted stimulation settings for the stimulation signal using a trained machine learning model, comprising generating a duration of stimulation cycle in the set of predicted stimulation settings by applying the trained machine learning model to a historical respiratory waveform of the person independent of a real-time respiratory waveform of the person;
providing the set of predicted stimulation settings to a stimulation signal generator; and
delivering the stimulation signal to the implantable electrode,
wherein the stimulation signal has a series of stimulation cycles each including a stimulation period and a non-stimulation period.

13. The method of claim 12, further comprising training a machine learning model using historical stimulation settings and historical patient physiological parameters to obtain the trained machine learning model.

14. The method of claim 12, wherein each stimulation setting of the set of predicted stimulation settings includes a stimulation parameter and a corresponding value for the stimulation parameter.

15. The method of claim 12, further comprising detecting one or more physiological parameters including an apnea-hypopnea index (AHI), a posture change, a sleep stage, and a time of day.

16. The method of claim 15, further comprising evaluating the set of predicted stimulation settings based at least in part on the one or more physiological parameters.

17. The method of claim 12, wherein:
the implantable electrode is a first implantable electrode,
the stimulation signal is a first stimulation signal to activate at least one first muscle for an upper airway dilation of the person, and
the method further comprises:
providing a second implantable electrode configured to deliver a second stimulation signal proximate to a second nerve to stimulate the second nerve and activate at least one second muscle for a caudal tracheal traction for an upper airway of the person;
generating a second set of predicted stimulation settings for the second stimulation signal using the trained machine learning model;
providing the second set of predicted stimulation settings to the stimulation signal generator; and
coordinating the delivery of the first stimulation signal with the delivery of the second stimulation signal.

18. The method of claim 12, wherein:
the implantable electrode is a first implantable electrode to a first stimulation signal proximate to a first nerve of the person to stimulate the first nerve and activate at least one first muscle for a caudal tracheal traction for an upper airway of the person;
the method further comprises:
providing a second implantable electrode configured to deliver a second stimulation signal proximate to a second nerve of the person to stimulate the second nerve and activate at least one second muscle for an upper airway dilation of the person;
generating a second set of predicted stimulation settings for the second stimulation signal using the trained machine learning model;
providing the second set of predicted stimulation settings to the stimulation signal generator; and
coordinating the delivery of the first stimulation signal with the delivery of the second stimulation signal.

19. The method of claim 12, wherein the implantable electrode is configured to deliver the stimulation signal proximate to a hypoglossal nerve to stimulate the hypoglossal nerve and activate at least one tongue muscle.

20. The method of claim 12, wherein the implantable electrode is configured to deliver the stimulation signal proximate to an ansa cervicalis nerve to stimulate the ansa cervicalis nerve and activate one or more infrahyoid muscles including sternothyroid and sternohyoid.
